(19) United States
(12) Patent Application Publication
(10) Pub. No.: US 2015/0345047 A1
(43) Pub. Date: Dec. 3, 2015

(54) SYSTEMS AND METHODS FOR IDENTIFYING DRUG COMBINATIONS FOR REDUCED DRUG RESISTANCE IN CANCER TREATMENT

(71) Applicant: Memorial Sloan Kettering Cancer Center, New York, NY (US)

(72) Inventors: Chris Sander, New York, NY (US); Anil Korkut, New York, NY (US)

(21) Appl. No.: 14/722,733

(22) Filed: May 27, 2015

Related U.S. Application Data
(60) Provisional application No. 62/004,480, filed on May 29, 2014.
(60) Provisional application No. 62/006,802, filed on Jun. 2, 2014.
(60) Provisional application No. 62/004,607, filed on May 29, 2014.
(60) Provisional application No. 62/006,804, filed on Jun. 2, 2014.

(51) Int. Cl. C40B 30/02; G01N 33/50

(52) U.S. Cl. CPC C40B 30/02; G01N 33/5011

(57) ABSTRACT

Methods and systems are presented herein for creating and using cell type-specific, quantitative network models of signaling in cells, such as melanoma, to predict cellular response to untested combinational perturbations. The methods involve performing a set of perturbation experiments with cells of a particular type to produce phosphoproteomic and/or phenotypic profiles for the cells; automatically extracting prior pathway information from one or more known databases to build a qualitative prior model; building a signaling pathway model from (i) the phosphoproteomic and/or phenotypic profiles produced from the perturbation experiments and (ii) the qualitative prior model from the known database(s); and performing in silico perturbations using the signaling pathway model to predict responses to a set of perturbation conditions not yet experimentally tested, and identifying one or more candidate drug combinations from the predicted responses.

### RELATED APPLICATIONS

This application claims the benefit of U.S. Provisional Patent Application Nos. 62/004,607 and 62/006,804, filed on May 29, 2014 and Jun. 2, 2014, respectively, and titled “Drug Combinations for Treatment of Melanoma and other Cancers,” and U.S. Provisional Patent Application Nos. 62/004,480 and 62/006,802, filed on May 29, 2014 and Jun. 2, 2014, respectively, and titled “Systems and Methods for Identifying Drug Combinations for Reduced Drug Resistance in Cancer Treatment,” the disclosures of which are incorporated herein by reference in their entireties.

### FIELD OF THE INVENTION

This invention relates generally to systems and methods for identifying drug combinations for treatment of cancer, and more particularly a cell type-specific, quantitative network model of signaling in cells (e.g., melanoma) that predicts cellular response to untested combinatorial perturbations.

### BACKGROUND

Inhibiting key oncogenic molecules with target specific agents elicit dramatic initial response in some cancers such as prostate cancer and melanoma. However, even for the most successful single agent targeted therapies, drug resistance eventually emerges, thus leading to rapid progression of metastatic disease. The mechanism of drug resistance may involve selection of resistant subclones, emergence of additional genomic alterations, and compensation of interactions between alternative signaling pathways.

Targeted therapy has been particularly successful in treatment of melanoma. BRAFV600E gain-of-function mutation is observed in ˜50% of melanomas. Direct inhibition of the BRAFV600E by RAF inhibitor (RAFi) vemurafenib has yielded response rates of more than 50% in melanoma patients with the mutation. Resistance to vemurafenib emerges after a period of ˜7 months in tumors that initially responded to the therapy. Multiple RAFi resistance mechanisms, which may involve alterations in RAF/MEK/ERK pathway (e.g., NRAS mutations, switching between RAF isoforms) or parallel pathways (e.g., PTEN loss), have been discovered in melanoma. These alterations may exist alone, in combinations, or emerge sequentially in a tumor.

One potential solution to overcome drug resistance is to combine targeted drugs to block escape routes. However, in order to systematically nominate drug combinations, there is a need for system-wide models that cover interactions between tens to hundreds of signaling entities and can describe and predict cellular response to multiple interventions. Prediction of cellular response to external perturbations is a central problem in biology. Qualitative interrogation of individual cellular components and processes by classical molecular biology techniques has limited predictive power. Comprehensive and quantitative models that link biomolecular and cellular response to external perturbations are potentially predictive. However, construction of such models is highly challenging due to the massively combinatorial nature of the biological processes, such as cellular signaling. Thus, there is also a need for improved systematic strategies to develop effective drug combinations.

### SUMMARY

Presented herein are methods and systems for creating and using cell typespecific, quantitative network models of signaling in cells, such as melanoma, to predict cellular response to untested combinatorial perturbations. The methods involve performing a set of perturbation experiments with cells of a particular type to produce phosphoproteomic and/or phenotypic profiles for the cells; automatically extracting prior pathway information from one or more known databases to build a qualitative prior model; building a signaling pathway model from (i) the phosphoproteomic and/or phenotypic profiles produced from the perturbation experiments and (ii) the qualitative prior model from the known database(s); and performing in silico perturbations using the signaling pathway model to predict responses to a set of perturbation conditions not yet experimentally tested, and identifying one or more candidate drug combinations from the predicted responses.

The (phospho)proteomic and phenotypic response profiles to paired targeted perturbations serve as the input for network inference. In an important example described herein in detail, the models capture the interactions between elements of multiple signaling pathways and phenotypes in the RAF inhibitor resistant melanoma cell line, SkMel133, which carries the BRAFV600E mutation and the homozygous PTEN and CDKN2A deletions. The resulting network models have high predictive power as shown with cross validation calculations. Through quantitative simulations, the inventors have predicted cellular response to tens of thousands of untested perturbation combinations. Guided by the predictions, they experimentally validated that co-targeting c-Myc using the BET bromodomain inhibitor, JQ1, and the RAF/MEK/ERK pathway using the kinase inhibitors leads to synergistic responses to overcome RAF inhibitor resistance in melanoma cells. The network modeling strategy provides a method of quantitative cell biology with particular emphasis on signaling interactions and prediction of cellular response to external interventions.

In one aspect, the invention is directed to a method for identifying a combination of two or more drugs the method comprising the steps of: (a) performing perturbation experiments whereby cells of a particular type are exposed to combinations of targeted compounds and high-throughput measurements of response profiles are performed to produce phosphoproteomic and/or phenotypic profiles from said perturbation experiments; (b) automatically extracting, by a processor of a computing device, prior signaling information from one or more databases and generating a qualitative prior model, wherein the qualitative prior model comprises a network of known interactions between proteins of interest; (c) constructing, by the processor, a network model of signaling using the phosphoproteomic and/or phenotypic profiles from step (a) and the qualitative prior model from step (b); (d) performing, by the processor, in silico perturbations using the network model of signaling from step (c) to predict responses to perturbation conditions not yet experimentally tested; and (e) identifying, by the processor, using the predicted responses of step (d), a candidate combination of two or more drugs.

In certain embodiments, the combination of two or more drugs is for treatment of cancer. In certain embodiments, the particular type of cells are cancer cells. In certain embodiments, (b) further comprises using a prior extraction and reduction algorithm. In certain embodiments, the proteins of interest are phosphoproteins profiled in step (a). In certain embodiments, the network model of signaling is an ODE-based signaling pathway model. In certain embodiments, the candidate combinations of two or more drugs is for treatment of cancer of the particular type used in the perturbation experiments of step (a).

In certain embodiments, the method further comprises the steps of: (e) performing additional experimental tests based on the predicted responses of step (d); and (f) identifying candidate drug combinations based on results of the additional experimental tests in step (e).

In another aspect, the invention is directed to a method for predicting responses to perturbation conditions to identify candidate drug combinations, the method comprising: (a) constructing, by a processor of a computing device, a network model of signaling using (i) a phosphoproteomic and/or phenotypic profiles and (ii) a qualitative prior model, wherein the phosphoproteomic and/or phenotypic profiles having been produced from perturbation experiments in which cells of a particular type are exposed to combinations of targeted compounds and high-throughput measurements of response profiles are performed to produce said phosphoproteomic and/or phenotypic profiles, and wherein the qualitative prior model comprising a network of known interactions between proteins of interest generated from one or more databases; (b) performing, by the processor, in silico perturbations using the network model of signaling to predict responses to perturbation conditions not yet experimentally tested; and (c) identifying, by the processor, a candidate combination of two or more drugs using the predicted responses of step (b).

In certain embodiments, the network model of signaling is an ODE-based signaling pathway model. In certain embodiments, the particular type of cells are cancer cells. In certain embodiments, the candidate combination of two or more drugs is for treatment of cancer of the type used in the perturbation experiments.

In another aspect, the invention is directed to a system for predicting responses to perturbation conditions to identify candidate drug combinations, the system comprising: a processor; and a memory having instructions stored thereon, wherein the instructions, when executed by the processor, cause the processor to: construct a network model of signaling using (i) a phosphoproteomic and/or phenotypic profiles and (ii) a qualitative prior model, wherein the phosphoproteomic and/or phenotypic profiles having been produced from perturbation experiments in which cells of a particular type are exposed to combinations of targeted compounds and high-throughput measurements of response profiles are performed to produce said phosphoproteomic and/or phenotypic profiles, and wherein the qualitative prior model comprising a network of known interactions between proteins of interest generated from one or more databases; perform in silico perturbations using the network model of signaling to predict responses to perturbation conditions not yet experimentally tested; and identify a candidate combination of two or more drugs using the predicted responses.

In certain embodiments, the network model of signaling is an ODE-based signaling pathway model. In certain embodiments, the particular type of cells are cancer cells. In certain embodiments, the candidate combination of two of more drugs is for treatment of cancer of the type used in the perturbation experiments.

Elements of embodiments described with respect to a given aspect of the invention may be used in various embodiments of another aspect of the invention. For example, it is contemplated that features of dependent claims depending from one independent claim can be used in apparatus and/or methods of any of the other independent claims.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example, streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

As used herein, for example, within the claims, the term “ligand” encompasses moieties that are associated with another entity, such as a nanogel polymer, for example. Thus, a ligand of a nanogel polymer can be chemically bound to, physically attached to, or physically entrapped within, the nanogel polymer, for example.

“Combination Therapy”: As used herein, the term “combination therapy”, refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

“Pharmaceutically acceptable”: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

“Pharmaceutical composition”: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Protein”: As used herein, the term “protein” refers to a polypeptide (i.e., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or comprises a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain Lamino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may comprise natural amino acids, non-natural amino acids, synthetic amino acids, and/or combinations thereof. In some embodiments, proteins are or comprise antibodies, antibody polypeptides, antibody fragments, biologically active portions thereof, and/or characteristic portions thereof.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polypeptide”: The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edurdadgrp/Unnatstruct.gif, which displays structures of non-natural amino acids that have been successfully incorporated into functional ion channels) and/or amino acid analogs as are known in the art may alternatively be employed). In some embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Subject”: As used herein, the term “subject” includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Drawings are presented herein for illustration purposes only, not for limitation.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The foregoing and other objects, aspects, features, and advantages of the present disclosure will become more apparent and better understood by referring to the following description taken in conjunction with the accompanying drawings, in which:

FIG. 1 illustrates quantitative and predictive signaling models generated from experimental response profiles to perturbations.

FIGS. 2A-2B illustrates response of melanoma cells to systematic perturbations with targeted agents. Melanoma (SkMel133) cells are perturbed using a set of targeted agents, seen in FIG. 2A. The melanoma cells are perturbed with combinations of targeted drugs as shown in the perturbation matrix (Table 3), shown in FIG. 2B.

FIGS. 3A-3H illustrate a use of prior information increases the predictive power of models.

FIGS. 3A-3D depict prior information is used for network inference.

FIGS. 3E-3H show no prior information is used for network inference.

FIGS. 4A-4D illustrate inferred network models capturing oncogenic signaling pathways in melanoma A.

FIG. 4A illustrates an average network model.

FIG. 4B illustrates a cell cycle oncogenic signaling pathway in melanoma A.

FIG. 4C illustrates a MAPK oncogenic signaling pathway in melanoma A.

FIG. 4D illustrates PI3K/AKT oncogenic signaling pathway in melanoma A.

FIGS. 5A-5H illustrate simulations with in silico perturbations provide predictions on system response to novel perturbations.

FIG. 5A depicts a schematic description of network simulations described in some embodiments herein.

FIG. 5B depicts model equations that are executed until all model variables (e.g., protein and phenotype responses) reach steady state.

FIG. 5C depicts simulations that expand the response map by three orders of magnitude and generate testable hypotheses.

FIGS. 5D-5H depict the top ten most effective single-agent in silico perturbations for each phenotype. For complete prediction heat maps for phenotypes, see FIGS. 13-14.

FIGS. 6A-6E illustrate the combined targeting of c-Myc with MEK and BRAF leads to synergistic response in melanoma cells.

FIG. 6A depicts the isobolograms of predicted G1-response to combined targeting of c-Myc with MEK, BRAF, CyclinD1 and pJUNpS73. The leftward shift of isocurves implies synergistic interactions between the applied perturbations. u denotes strength of in silico perturbations.

FIG. 6B depicts western blots showing the level of BRD4 (top) 24 hours after JQ1 treatment. The BRD4 level is unchanged in response to JQ1 treatment and western blots showing the expression levels of c-Myc (bottom) in response to JQ1, MEKi, RAFi and their combinations 24 hours after drug treatment. c-Myc expression is targeted with BRD4 inhibitor, JQ1.

FIG. 6C depicts the cell viability drug dose-response curves for MEKi and JQ1. Cell viability is measured using the resazurin assay (top) and the cell viability drug dose-response curves for RAFi and JQ1 (bottom).

FIG. 6D depicts the synergistic interactions between JQ1 and RAFi/MEKi. The combination index (CI) calculated using the drug dose response curves quantifies the drug interactions between JQ1, MEKi and RAFi (left panel). CI for a given level of inhibition is a measure of the fractional shift between the combination doses (C1 and C2) and the single agent's inhibitory concentration (Cx,1, Cx,2). It is formulated as CI=(C1/Cx,1)+=(C2/Cx,2). Amax is the response of melanoma cells to JQ1, MEKi and RAFi at highest doses applied (Right panel). 1-Amax is the fraction of cells alive in response to highest drug dose normalized with respect to the non-drug treated condition.

FIG. 6E depicts the cell cycle progression phenotype in response to JQ1. 86% of cells are in G1 state 48 hours after JQ1 treatment, while 42% of the cells are in G1 state before drug treatment. Error bars: ±SEM in 3 biological replicates.

FIG. 7 illustrates BP-guided decimation algorithm is used to construct executable, individual network model solutions from BP generated probability distributions for each edge strength value (wij). The algorithm chart depicts one round of BP-guided decimation to generate a single model solution. Consecutive runs of BP-guided decimation algorithm leads to construction of a network model solution ensemble.

FIG. 8 illustrates cell cycle arrest response of melanoma cells to JQ1 and MEKi combination. 51% and 88% of cells are in G1 stage 24 hours after JQ1 and MEKi treatment, respectively. JQ1+MEKi combination has an increased effect on G1 cell cycle arrest (G1=92%). 39% of cells are in G1 when they are not treated with drugs. Error bars in right panel: ±SE in 3 biological replicates.

FIGS. 9A and 9B illustrate clustering and pathway analysis of proteomics data. The proteomic signatures were characterized in the response data with a pathway analysis guided by hierarchical clustering.

FIG. 9A shows the two-way clustering analysis of the experimental response map reveals distinct proteomic signatures of response to drugs targeting different signaling pathways.

FIG. 9B shows extracted signaling interactions between the proteomic entities that fell into each cluster.

FIG. 10 illustrates the prior model of signaling. The prior information model is extracted from Reactome and NCI PID using the PERA algorithm. The prior model served as a bias in BP-based network inference. Dashed arrows represent priors for activating edges, red arrows represent inhibitory edges and black arrows represent generic edges (e.g., activating or inhibitory).

FIG. 11 illustrates comparison of random vs. actual prior information.

FIGS. 12A and 12B illustrate distribution of edges in the solution ensemble. In order to model cellular signaling and predict response, 4000 signaling models were computed and used to generate a solution ensemble.

FIG. 12A shows edge frequencies (f(wᵢⱼ)) in the solution ensemble.

FIG. 12B shows the frequency distribution of nonzero edges (|wᵢⱼ|>0.2) has a bimodal character.

FIGS. 13A-13E illustrate prediction of phenotypic responses to in silico, combinatorial perturbations. For cell cycle phenotypes, the desired response is an increase in the cell cycle arrest (Darker grey: increased cell cycle arrest. Lighter grey: Decreased cell cycle arrest).

FIG. 13A illustrates prediction of cell viability responses to in silico, combinatorial perturbations.

FIG. 13B illustrates prediction of G1 arrest responses to in silico, combinatorial perturbations.

FIG. 13C illustrates prediction of S arrest responses to in silico, combinatorial perturbations.

FIG. 13D illustrates prediction of G2 arrest responses to in silico, combinatorial perturbations.

FIG. 13E illustrates prediction of G2-M arrest responses to in silico, combinatorial perturbations.

FIG. 14 illustrates changes in c-Myc level in response to perturbations in Skmel133 cells as measured in RPPA experiments. Each data point is log normalized with respect to the c-Myc level in unperturbed condition. c-Myc level is highest in the presence of CDK4i. Various drug combinations that include STAT3 and mTOR inhibitors follow CDK4i combinations. CMyc level is lowest when cells are perturbed with MEK, BRAF, SRC, PKC and HDAC inhibitors, respectively. Each data-point is the average of RPPA readouts from three replicates.

FIG. 15 is a block diagram of an example network environment for use in the methods and systems for analysis of spectrometry data, according to an illustrative embodiment.

FIG. 16 is a block diagram of an example computing device and an example mobile computing device, for use in illustrative embodiments of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

It is contemplated that articles, apparatus, methods, and processes of the claimed invention encompass variations and adaptations developed using information from the embodiments described herein. Adaptation and/or modification of the articles, apparatus, methods, and processes described herein may be performed by those of ordinary skill in the relevant art.

Throughout the description, where articles and apparatus are described as having, including, or comprising specific components, or where processes and methods are described as having, including, or comprising specific steps, it is contemplated that, additionally, there are articles and apparatus of the present invention that consist essentially of, or consist of, the recited components, and that there are processes and methods according to the present invention that consist essentially of, or consist of, the recited processing steps.

It should be understood that the order of steps or order for performing certain actions is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Network Models and their Complexity

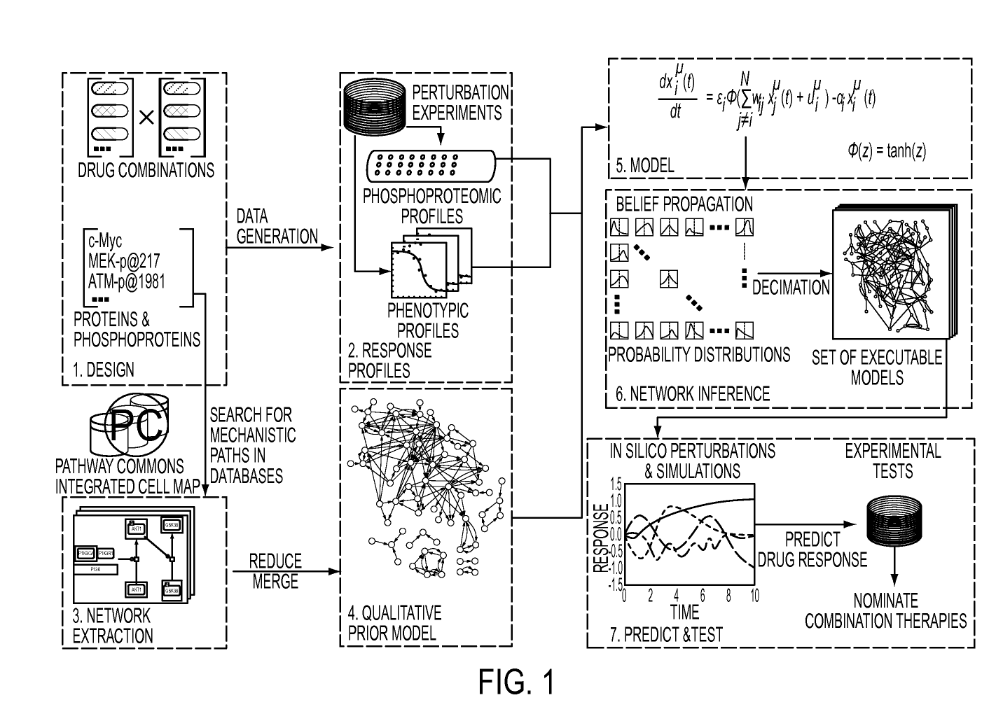

Provided herein are methods of constructing system-wide signaling models that link drug perturbations, (phospho)proteomic changes and phenotypic outcomes (FIG. 1). As shown in FIG. 1, quantitative and predictive signaling models are generated from experimental response profiles to perturbations. Perturbation biology involves systematic perturbations of cells with combinations of targeted compounds (FIG. 1, Box 1 (Design) and Box 2 (Response profiles)), high-throughput measurements of response profiles (FIG. 1, Box 2 (Response profiles)), automated extraction of prior signaling information from databases (FIG. 1, Box 3 (Network extraction) and Box 4 (Qualitative prior model)), construction of ODE-based signaling pathway models (FIG. 1, Box 5 (Model)) with the belief propagation (BP) based network inference algorithm (FIG. 1, Box 6 (Network Inference)), and prediction of system response to novel perturbations with the models and simulations (FIG. 1, Box 7 (Predict & Test)). The “prior extraction and reduction algorithm” (PERA) generates a qualitative prior model, which is a network of known interactions between the proteins of interest (e.g., profiled (phospho)proteins). This is achieved through a search in the Pathway Commons information resource, which integrates biological pathway information from multiple public databases. In the quantitative network models, the nodes represent measured levels of (phospho)proteins or cellular phenotypes, and the edges represent the influence of the upstream nodes on the time derivative of their downstream effectors. This approach corresponds to a simple yet efficient ODE-based mathematical description of models. The BP-based modeling approach combines information from the perturbation data (phosphoproteomic and phenotypic) with prior information to generate network models of signaling. The resulting ODE based models are executed to predict system response to untested perturbation conditions.

The system-wide signaling models capture dynamic signaling events and predict cellular response to previously untested combinatorial interventions. In order to generate the training data for network modeling, systematic perturbation experiments are first performed in cancer cells with targeted agents. The next step is profiling proteomic and phenotypic response of cells to the perturbations. The cell type specific response data serves as the input in network inference. Accurate signaling network inference requires sampling of models from a prohibitively large and complex search space. Therefore, prior pathway information from signaling databases is incorporated to narrow the parameter search space and improve the accuracy of the models. For this purpose, a computational tool is used (herein referred to as Pathway Extraction and Reduction Algorithm or “PERA”) to automatically extract priors from Pathway Commons. In network inference, prior information introduces soft-restraints on the search space (e.g., the algorithm rejects the prior information that does not conform to the experimental training data).

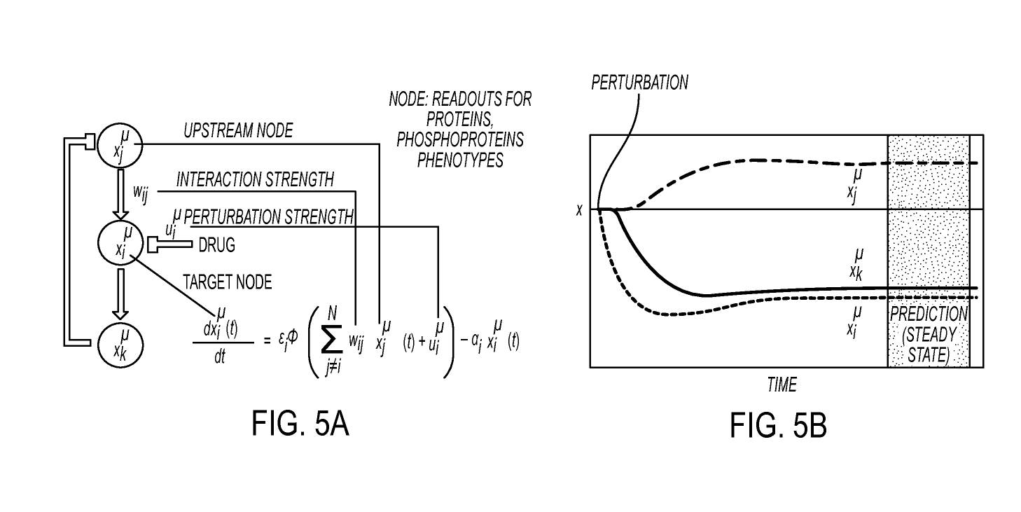

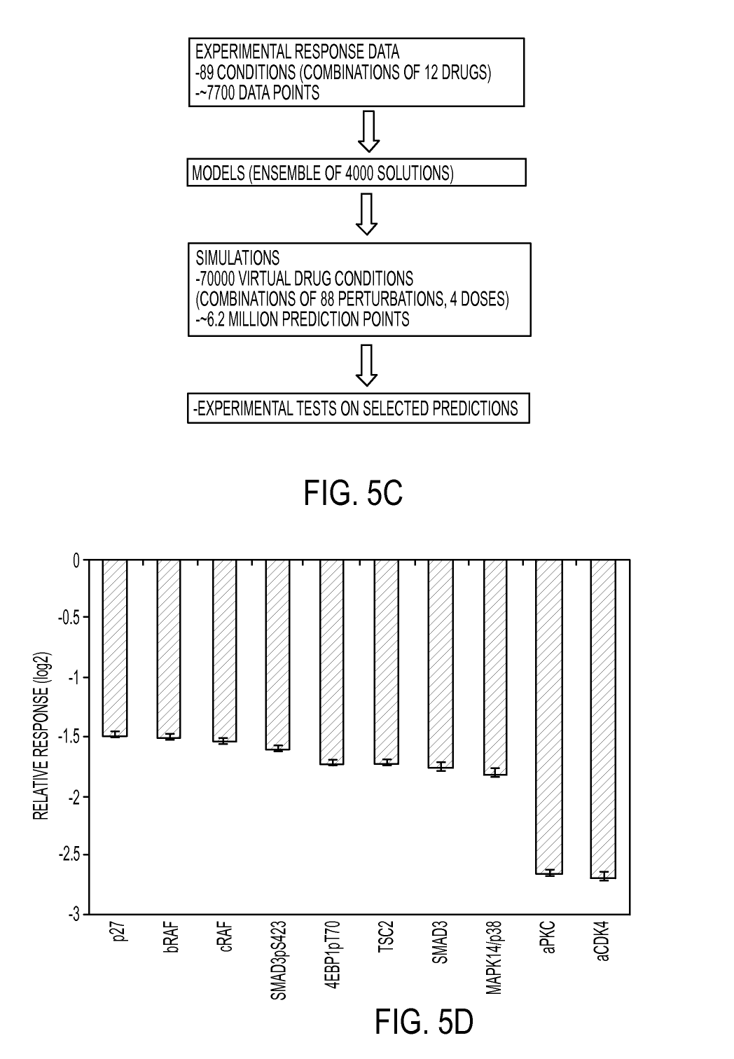

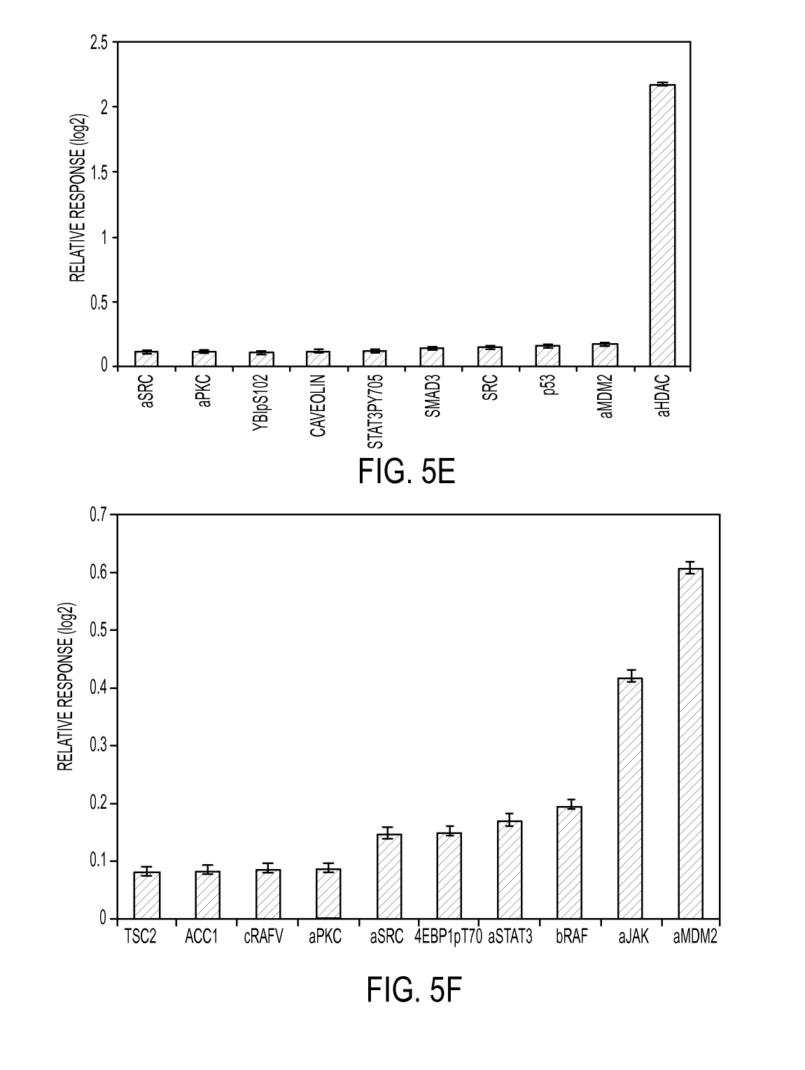

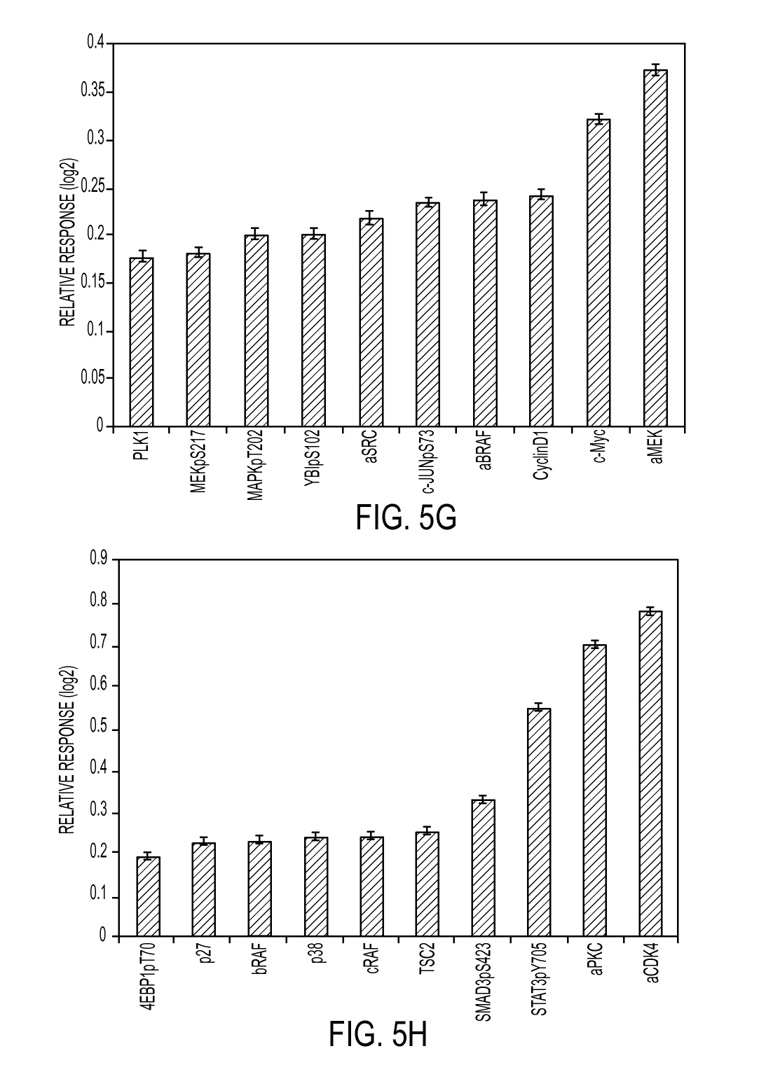

Even in the presence of large training data and priors, network inference is a difficult problem due to the combinatorial complexity (e.g., exponential expansion of the parameter search space with linear increase of parameters). The combinatorial complexity creates a practical limit on the size of the models that can be inferred non-heuristically. To circumvent this problem, a network modeling algorithm is adapted based on belief propagation (BP). The algorithm enables construction of cell type specific models that can predict response of hundreds of signaling entities to combinatorial perturbations. Using the network models, cellular response to untested combinatorial perturbations is predicted. For this purpose, the fully parameterized network models are simulated with in silico perturbations until the system reaches steady state (FIGS. 1 and 5A-H). The steady state readout for each proteomic and phenotypic entity (e.g., system variables) is the predicted response to the perturbations.

In an example described herein, cell type specific network models of signaling in RAFi resistant melanoma cells are constructed from perturbation experiments. The models quantitatively linked 94 proteomic nodes with 5 phenotypic nodes. As shown by cross validation calculations, use of prior information significantly improved the predictive power of the models. Once the predictive power was established, the extent of the drug response information was expanded from a few thousands experimental data points to millions of predicted points in melanoma cells. Based on the predictions, a candidate drug combination was identified (e.g., co-targeting c-Myc with BRAF or MEK was identified as a strategy to overcome RAFi drug resistance). First, the BET bromodomain inhibitor, JQ1, was experimentally shown to reduce c-Myc expression, and next, co-targeting c-Myc with RAF or MEK was found to lead to synergistic effects on the growth of RAFi resistant SkMe1-133 cells.

### Methods

### Cell Cultures and Perturbation Experiments

RAFi resistant melanoma cell line SkMel133 was used in all perturbation experiments. SkMel133 cells were perturbed with 12 targeted drugs applied as single agents or in paired combinations. Table 1 below shows a list of the drugs used in perturbation experiments.

TABLE 1

| Drug | | Downstream | Dose 1 | Dose 2 |
|---|---|---|---|---|
| name | Target | effector | (nM) | (nM) |
| AKTi 1/2 | AKT | AKTpS473 | 5.000 | 10.000 |
| Temsirolimus | mTOR | S6pS240 | 0.3 | 0.6 |
| ZSTK474 | PI3K | AKTpS473 | 600 | 1200 |
| PD0325901 | MEK1/2 | MAPK1pT202 | 1.5 | 3 |
| Stattic | STAT3 | STAT3pY705 | 600 | 1200 |
| HNHA | HDAC | P27/Kip1 | 6,000 | 12,000 |
| PLX4720 | BRAF^{V600E} | MAPK1pT202 | 60 | 120 |
| RO-31-7549 | PKC | S6pS240 | 3500 | 7000 |
| RY | CDK4 | Rb_pS807 | 2000 | NA |
| P6 | JAK | GSK3pS21 | 20 | 40 |
| SR | SRC | GSK3pS21 | 2,400 | 4,800 |
| Nutlin | MDM2 | 4EBP1pS65 | 3,000 | 6,000 |

### Quantitative Phenotypic Assays

All phenotypic measurements were made in perturbation conditions identical to those in proteomic measurements. Cell viability and cell cycle progression were measured 72 hours after drug treatment using the Resazurin assay and flow cytometry analysis respectively. The percentage of cells in the G1, G2/M, and S phases and sub-G1 fraction were recorded based on the respective distribution of DNA content in each phase.

### Reverse Phase Protein Arrays

Proteomic response profiles to perturbations were measured using reverse phase protein arrays. The cells were lysed 24 hours after drug treatment. Three biological replicates were spotted for each sample (e.g., drug condition) on RPPA slides. Each slide was stained with the respective Ab and 138 proteomic entities (total or phosho levels) and were profiled using specific Abs with the RPPA (Table 3).

Automated Extraction of Prior Information from Signaling Databases

A software tool (PERA) was used to automatically extract prior information from multiple signaling databases and generate a prior information network. The input to PERA was a list of (phospho) proteins identified by their HGNC symbols (e.g. AKT1), phosphorylation sites (e.g. pS473), and their molecular status (e.g., activating or inhibitory phosphorylation, total concentration). The output of PERA was a set of directed interactions between signaling molecules represented in a Simple Interaction Format (SIF). Table 2 below is a list of proteins used in modeling.

TABLE 2

| Protein | Phosphorylation | Protein | Phosphorylation |
|---|---|---|---|
| 4EBP1 | S65 | IG1Rβ | |
| 4EBP1 | T37 | IGFBP2 | |
| 4EBP1 | T70 | IRS1 | S307 |
| α-Tubulin | | IRS1 | |
| ACC1 | S79 | ERK/MAPK1 | T202 |
| ACC1 | | MEK | S217 |
| AKT | S473 | mTOR | S2448 |
| AKT | T308 | p21/Cip1 | |
| AKT | | p27/Kip1 | |
| AMPK | T172 | p38/MAPK14 | T180 |
| ATM | S1981 | p38/MAPK14 | |
| ATR | | P53 | |
| β-Catenin | S33 | p70S6K | T389 |
| β-Catenin | | PAI-1 | |
| BAK | | PAX2 | |
| BCL-XL | | PCNA | |
| BCL2 | | PDK1 | S241 |
| BIM | | PI3Kp85 | |
| BRAF | | PKCa | |
| c-JUN | S73 | PLK1 | |
| c-Myc | | RAD51 | |
| Caspase9 | | Rb | S807 |
| Caspase9clvdAsp31 | | S6 | S235 |
| Caveolin | | S6 | S240 |
| CHK1 | S345 | S6 | |
| CHK2 | T68 | SMAD3 | S423 |
| Collagenase | | SMAD3 | |
| COX2 | | SRC | Y527 |
| cRAF | | SRC | Y416 |
| CyclinB1 | | SRC | |
| CyclinD1 | | STAT3 | Y705 |
| CyclinE1 | | STAT3 | |
| EGFR | | STAT5 | Y694 |
| ELK1 | S383 | STAT5 | |
| ER-α | | STAT6 | Y641 |
| Fibronectin | | TAZ | S89 |
| GATA3 | | TSC2 | T1462 |
| GSK3-αβ | S21 | TSC2 | |
| GSK3-αβ | S9 | XRCC1 | |
| GSK3-αβ | | YAP | S127 |
| HSP27 | | YBI | S102 |

### Mathematical Description of Network Models

The network models represent the time behavior of the cellular system in a set of perturbation conditions as a series of coupled nonlinear ordinary differential equations (ODE).

$$\begin{matrix}Network model ODEs & \\ \frac{d {x}_{i}^{μ} ( t )}{d t} = {ɛ}_{i} φ ( {\sum }_{j \neq i}^{N} {w}_{ij} {x}_{j}^{μ} ( t ) + {u}_{i}^{μ} ) - {α}_{i} {x}_{i}^{μ} ( t ) \\ φ ( z ) = tanh ( z ) & Equation 1\end{matrix}$$

In the network models, each node represents the quantitative change of a biological variable, xᵢ^{μ} (e.g., total or phosphoprotein level and phenotypic change) in the perturbed condition, μ relative to the unperturbed condition. Wᵢⱼ quantifies the edge strength, which is the impact of upstream node j on the time derivative of downstream node i. A semi-discreet values is assigned to each Wᵢⱼ. W={wᵢⱼ, ∀wᵢⱼε{−1, −0.8, . . . 0.8, 1}}. αᵢ constant is the tendency of the system to return to the initial state, and εᵢ constant defines the dynamic range of each variable i. The transfer function Φ(x) ensures that each variable has a sigmoidal temporal behavior.

Modified Cost Function for Network Inference with Prior Information

The cost of a model solution was quantified by an objective cost function C(W). The network configurations with low cost represent the experimental data more accurately. Here, an additional prior information term was incorporated to the cost function to construct models with improved predictive power. The newly introduced term in the cost function accounts for the prize introduced when the inferred wᵢⱼ is consistent with the prior information. The modified cost function with prior information term is formulated as Equation 2.

$$\begin{matrix} Modified Cost fuction & Equation 2 \\ \begin{matrix} C ( W ) = {C}^{SSE} ( W ) + {C}^{complexity} ( W ) + {C}^{prior} ( W ) \\ C ( W ) = β {\sum }_{1}^{L} {\sum }_{i}^{N} {\sum }_{μ}^{M} {( {x}_{i}^{μ} ( {t}_{1} ) - {x}_{i}^{μ *} ( {t}_{1} ) )}^{2} + λ {\sum }_{i}^{N} {\sum }_{j \neq i}^{N} δ ( {w}_{ij} ) + η ( W )\end{matrix} & 2. a \\ \begin{matrix} δ ( {w}_{ij} ) = 1 & if δ ( {w}_{ij} ) \neq 0 \\ δ ( {w}_{ij} ) = 0 & if δ ( {w}_{ij} ) = 0\end{matrix} & 2. b\end{matrix}$$

With reference to Equation 2, the first term penalizes the discrepancies between predicted xᵢ^{μ} and experimental xᵢ^{μ*} values of the system variables at a time points t₁ in condition μ. The second term is the complexity factor with an L0 norm, which reduces the number of nonzero interactions in a network configuration and ensures that resulting network models are sparse. The final term, η(W) is the prior cost function of W={wᵢⱼ}. η(wᵢⱼ=ω) has a negative real value if the wᵢⱼ=ω is included in the prior model. For each interaction in the prior information network, η(wᵢⱼ=ω) has a negative value. Therefore, the prior information introduces a prize that reduces the overall cost function. The model error and complexity terms are identical to those previously reported. Here the newly introduced prior information term is formulated in the modified cost function.

The prior information from databases may represent direct or logical interactions between the proteomic entities in similar nature to the interactions in the inferred network models. The prior information may refer to activating (positive signed wᵢⱼ), inhibitory (negative signed wᵢⱼ), or generic (e.g., no preference for a sign in priors) interactions. A generalized prior information cost term has been formulated, which samples the prior prize from a Gaussian distribution. Described herein, a simplified, binary form of the prior term was used, since state of the art signaling databases provide only binary interactions. The binary nature of the interactions implies a generic weight (κ) for each interaction represented in prior information network. The resulting prior information term in the cost function is a step function.

η(wᵢⱼ≠0)=−κ and η(wᵢⱼ=0)=0 Generic prior information (wᵢⱼ≠0)

η(wᵢⱼ>0)=−κ and η(wᵢⱼ≦0)=0 Prior information for an activating interaction (wᵢⱼ>0)

η(wᵢⱼ<0)=−κ and η(wᵢⱼ≧0)=0 Prior information for an inhibitory interaction (wᵢⱼ<0)

η(wᵢⱼ)=0 No Prior exists for w Equation 3: Simplified Error model of individual prior observations

Finally, the cumulative prior prize for W={wᵢⱼ} in Equation 2 becomes Equation 4.

$$\begin{matrix}Simplified cumulative error \\ model of prior observations & \\ η ( W ) = {\sum }_{i}^{N} {\sum }_{j}^{N} η ( {w}_{ij} ) & Equation 4\end{matrix}$$

A generalized form of the prior information term was also described, which incorporates each prior as a Gaussian distributed bias.

### Network Model Construction and Response Prediction

Network models are constructed with a two-step strategy. The method is based on first calculating probability distributions for each possible interaction at steady-state with the Belief Propagation (BP) algorithm and then computing distinct solutions by sampling the probability distributions. The theoretical formulation was described, the underlying assumptions and simplification steps of the BP algorithm for inferring network models of signaling elsewhere. The network models include 82 proteomic, 5 phenotypic and 12 activity nodes. Activity nodes couple the effect of drug perturbations to the overall network models.

### Belief Propagation

Belief propagation algorithm iteratively approximates the probability distributions of individual parameters. The iterative algorithm is initiated with a set of random probability distributions. In each iteration step, individual model parameters are updated (e.g., local updates) based on the approximate knowledge of other parameters, experimental constraints and prior information (e.g., global information). In the next iteration, the updated local information becomes part of the global information and another local update is executed on a different model parameter. The successive iterations continue over different individual parameters until the updated probability distributions converge to stable distributions. The iterations between the local updates and the global information create an optimization scheme that W={wᵢⱼ} is inferred given a probability model. Explicitly, the following cavity update equations are iteratively calculated until convergence.

$$\begin{matrix}BP update equation & \\ {P}^{μ} ( {w}_{ij} ) = \frac{1}{{Z}_{ij}} {e}^{- λδ ( {w}_{ij} )} . {e}^{η ( {w}_{ij} )} {\prod }_{υ \neq μ}^{M} {ρ}^{υ} ( {w}_{ij} ) ∀ j \neq k & Equation 5 a \\ BP update equation & \\ {ρ}^{μ} ( {w}_{ik} = ω ) = \frac{1}{{Z}_{ik}} {\sum }_{{w}_{ij} , j \neq k}^{ } {e}^{- {β ( {x}_{i}^{μ} - {x}_{i}^{μ *} )}^{2}} {\prod }_{j , j \neq k}^{N} {P}^{μ} ( {w}_{ij} ) & Equation 5 b\end{matrix}$$

In Equation 5a, P^{μ}(wᵢⱼ) approximates the mean field of the parameters with a sparsity constraint (λδ(wᵢⱼ)) and a bias from prior information restraints (η(wᵢⱼ)). In Equation 5b, ρ^{μ}(wᵢⱼ=ω) is a mean field derived change to the probability distribution of the locally optimized parameter, towards minimizing the model error (C^{SSE}(W)).

### BP-Guided Decimation

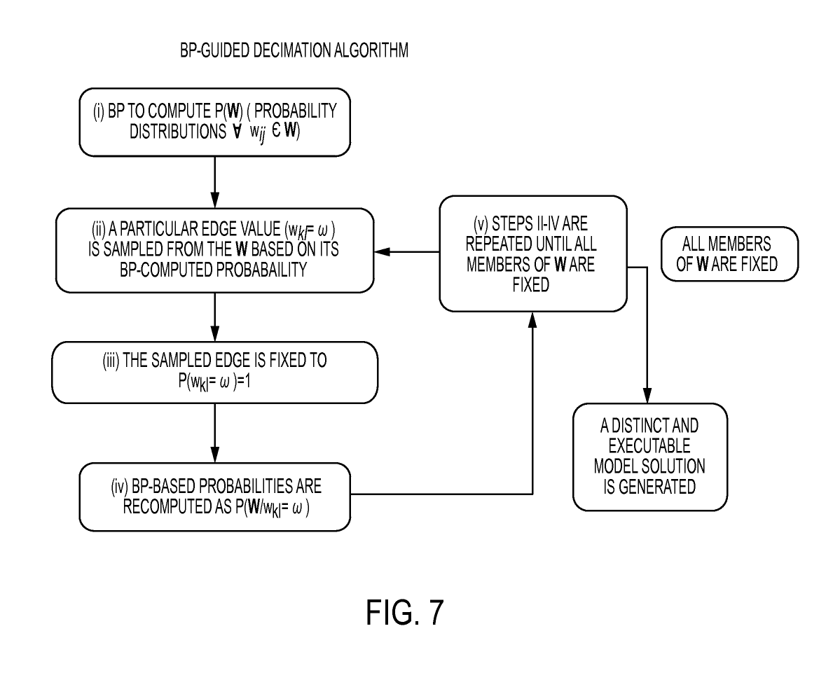

Distinct network models are instantiated from BP generated probability distributions with the BP-guided decimation algorithm (FIG. 7). This procedure generates distinct and executable network models. As described herein, 4000 distinct network models are generated in each computation.

More specifically, FIG. 7 illustrates how the BP-guided decimation algorithm is used to construct executable, individual network model solutions from BP generated probability distributions for each edge strength value (wij). The algorithm chart of FIG. 7 depicts one round of BP-guided decimation to generate a single model solution. Consecutive runs of BP-guided decimation algorithm leads to construction of a network model solution ensemble.

Simulations with in Silico Perturbations

Network models are executed with specific in silico perturbations until all system variables {xᵢ} reach steady state. The perturbations acting on node i are exerted as real-valued uᵢ^{μ} vectors in model Equation 1. The DLSODE integration method (ODEPACK) is used in simulations (default settings with, MF=10, ATOL=1e-10, RTOL=1e-20).

### Examples

### 1. Experimental Response Maps from Proteomic/Phenotypic Profiling

### Drug Perturbation Experiments in Melanoma Cells

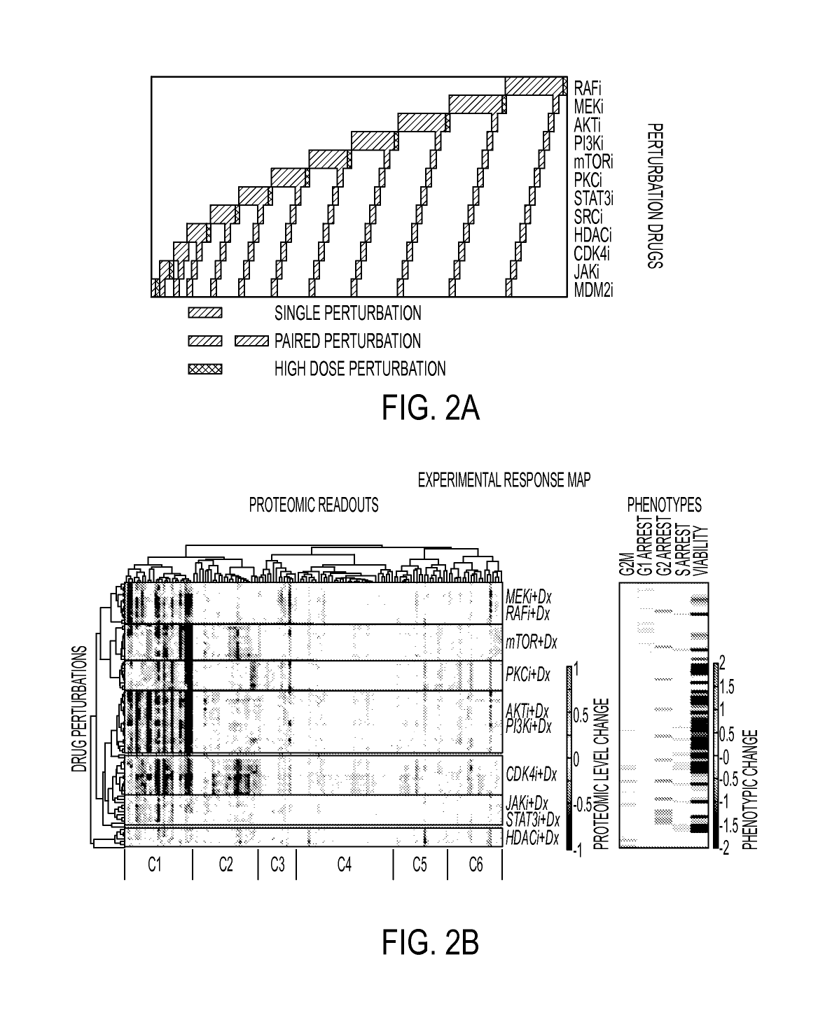

Systematic perturbation experiments were performed in malignant melanoma cells (FIG. 2A) to generate a rich training set for network inference. FIGS. 2A and 2B illustrate responses of melanoma cells to systematic perturbations with targeted agents. More specifically, FIG. 2A shows perturbation of melanoma (SkMel133) cells using a set of targeted agents. FIG. 2B shows a perturbation matrix of perturbation of the melanoma cells with combinations of targeted drugs.

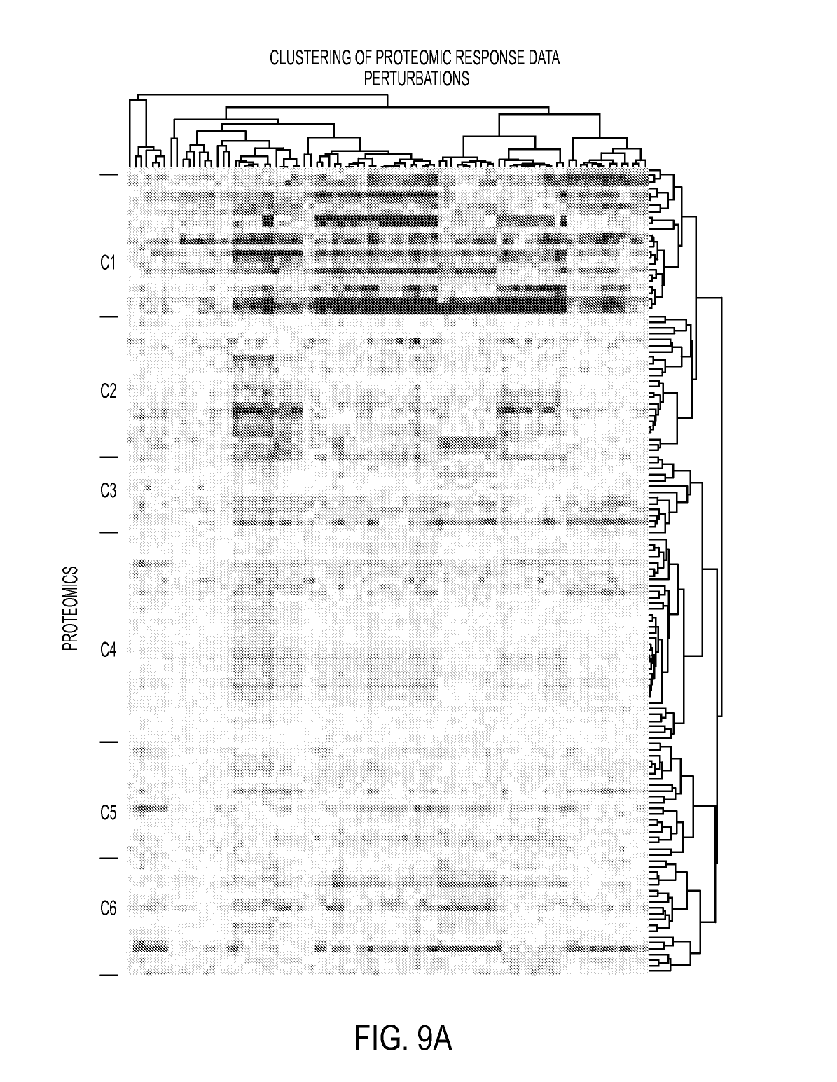

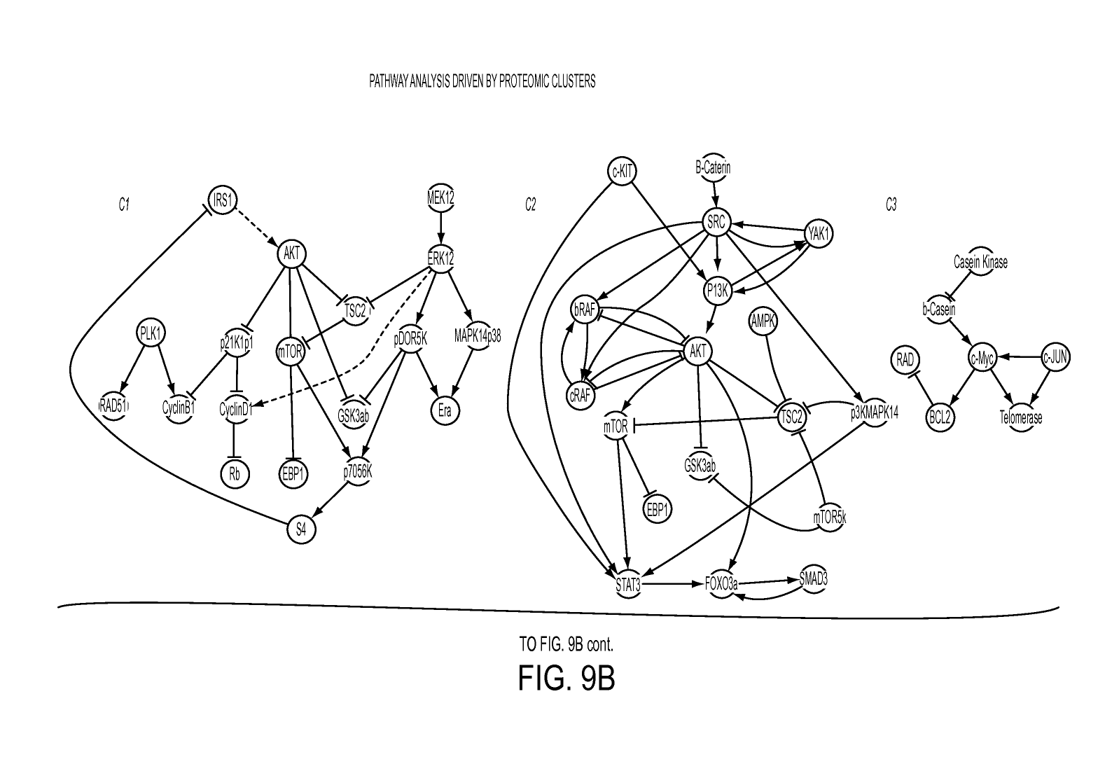

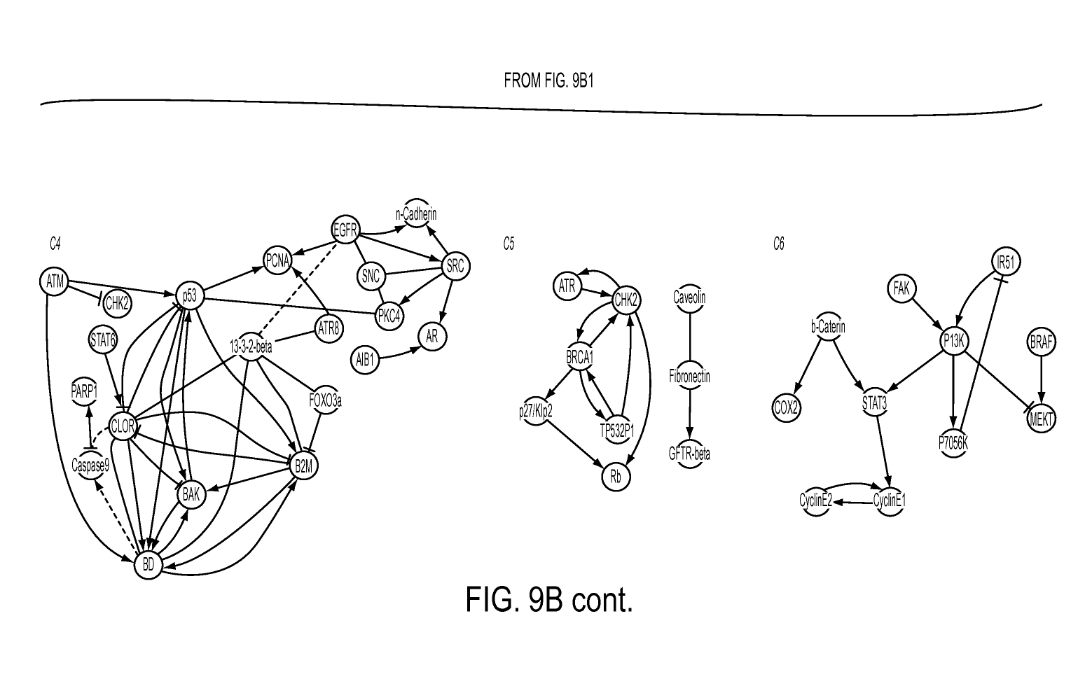

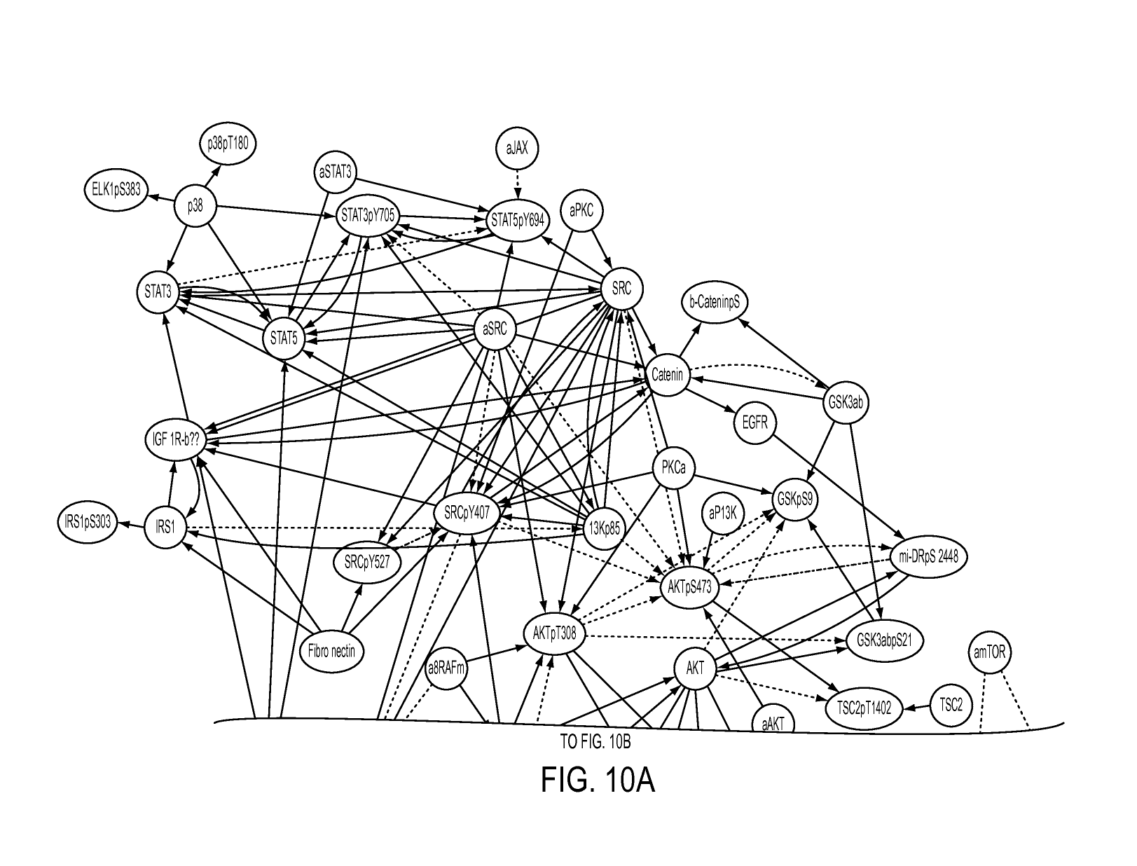

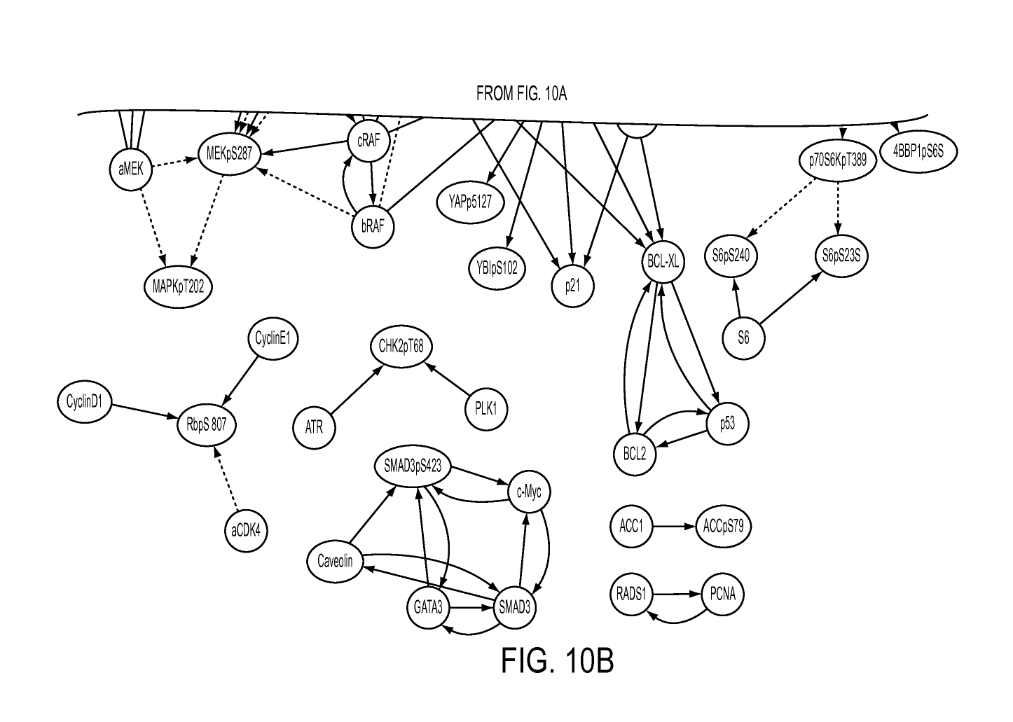

The concentration changes in 138 proteomic entities (FIG. 2B, left) and the phenotypic changes (FIG. 2B, right) in response to drug combinations with respect to the untreated conditions formed an experimental “response map” of the cellular system. The response map reflected the functional relations between signaling proteins and cellular processes (FIG. 10). The two-way clustering analysis of the proteomic readouts revealed distinct proteomic response signatures for each targeted drug (FIGS. 9A and 9B). Cell cycle progression and viability response was measured using flow cytometry and resazurin assays respectively. The cell cycle progression phenotype was quantified based on the percentage of the cells in a cell cycle state in perturbed condition with respect to the unperturbed condition. For the phenotypic readouts, the order of the perturbation conditions was same as in FIG. 9A. The response values were relative to a no drug control and given as log₂(perturbed/unperturbed).

More specifically, FIGS. 9A and 9B depict clustering and pathway analysis of proteomics data. The proteomic signatures in the response data was characterized with a pathway analysis guided by hierarchical clustering. FIG. 9A illustrates a two-way clustering analysis of the experimental response map revealing distinct proteomic signatures of response to drugs targeting different signaling pathways. The Cluster software was used for the two way hierarchical clustering with correlation-based distance metric and average-linkage method. Three major (C1, C2 and C4) and three minor (C3, C5, C6) proteomic response clusters were identified. FIG. 9B illustrates the extraction of signaling interactions between the proteomic entities that fell into each cluster. The signaling interactions within each proteomic cluster were extracted from Pathway Commons 2 database using the PERA algorithm. The pathway diagrams were simplified by removing the post-translational modifications and merging the nodes associated with identical genes (e.g., AKTpT308, AKTpS474 and AKT are merged to AKT node). The resulting diagrams displayed the known pathways associated with proteomic clusters, whose members gave similar response to targeted agents. Without being bound by theory, the cluster guided pathway analysis suggests that functionally related proteins (e.g., proteins on the same or related pathways) are enriched in distinct clusters. The phospho-proteomic entities on MAPK and PI3K/AKT pathways are enriched in cluster 1 (C1) of the response map. The total protein measurements related to MAPK and PI3K/AKT pathways were enriched in a related but distinct cluster (C2). The level of various apoptotic proteins increased in response to most targeted drugs and form another distinct cluster (C4). Interestingly, EGFR (Both phospho and total level) and a set of EGFR related docking proteins (e.g., SHC, 14-3-3-(3) were also enriched in C4 possibly due to the increase in the expression of those entities in response to targeted drugs such as MEKi and PI3Ki. Without being bound by theory, the observed increase in EGFR level suggests a potential feedback loop that emerges from downstream elements of MAPK and PI3K/AKT pathways, which were targeted by multiple agents as described herein.

RAFi resistant melanoma cell line SkMel133, which has the BRAFV600E mutation as well as homozygous PTEN and CDKN2A deletions, was treated with combinations of 12 targeted drugs (FIG. 2A, Table 1).

The perturbations consisted of systematic paired combinations of individual agents and multiple doses of single agents. This procedure generated 89 unique perturbation conditions, which targeted specific pathways including those important for melanoma tumorigenesis such as RAF/MEK/ERK and PI3K-AKT.

In total, cells were treated with 89 unique perturbations (Table 3). In paired combinations, each drug concentration was selected to inhibit the readout for the presumed target or the downstream effectors by 40% (IC40) as determined by Western Blot experiments. In single agent perturbations, each drug is applied at two different concentrations, IC40 and 2×IC40.

Table 3 below shows a list of the perturbation conditions.

TABLE 3

| Index | Perturbation1 | Dose1 (nM) | Perturbation2 | Dose2 (nM) |
|---|---|---|---|---|
| 1 | MEKi | 1.5 | | |
| 2 | MEKi | 1.5 | HDACi | 6,000 |
| 3 | MEKi | 1.5 | MDM2i | 3,000 |
| 4 | MEKi | 1.5 | JAKi | 20 |
| 5 | MEKi | 1.5 | BRAFi | 60 |
| 6 | MEKi | 1.5 | PKCi | 3500 |
| 7 | MEKi | 1.5 | SRCi | 2400 |
| 8 | MEKi | 1.5 | STAT3i | 600 |
| 9 | MEKi | 1.5 | mTORi | 0.3 |
| 10 | MEKi | 1.5 | PI3Ki | 600 |
| 11 | MEKi | 3 | | |
| 12 | AKTi | 10,000 | | |
| 13 | AKTi | 5,000 | | |
| 14 | AKTi | 5,000 | MEKi | 1.5 |
| 15 | AKTi | 5,000 | HDACi | 6000 |
| 16 | AKTi | 5,000 | MDM2i | 3000 |
| 17 | AKTi | 5,000 | JAKi | 20 |
| 18 | AKTi | 5,000 | BRAFi | 60 |
| 19 | AKTi | 5,000 | PKCi | 3500 |
| 20 | AKTi | 5,000 | SRCi | 2400 |
| 21 | AKTi | 5,000 | STAT3i | 600 |
| 22 | AKTi | 5,000 | mTORi | 0.3 |
| 23 | AKTi | 5,000 | PI3Ki | 600 |
| 24 | HDACi | 12,000 | | |
| 25 | HDACi | 6,000 | | |
| 26 | HDACi | 6,000 | MDM2i | 3000 |
| 27 | HDACi | 6,000 | PKCi | 3500 |
| 28 | HDACi | 6,000 | SRCi | 2400 |
| 29 | HDACi | 6,000 | mTORi | 0.3 |
| 30 | MDM2i | 3,000 | | |
| 31 | MDM2i | 3000 | PKCi | 3500 |
| 32 | MDM2i | 3000 | SRCi | 2400 |
| 33 | MDM2i | 3000 | mTORi | 0.3 |
| 34 | MDM2i | 6,000 | | |
| 35 | JAKi | 20 | | |
| 36 | JAKi | 20 | HDACi | 6000 |
| 37 | JAKi | 20 | MDM2i | 3000 |
| 38 | JAKi | 20 | PKCi | 3500 |
| 39 | JAKi | 20 | SRCi | 2400 |
| 40 | JAKi | 20 | STAT3i | 600 |
| 41 | JAKi | 20 | mTORi | 0.3 |
| 42 | JAKi | 40 | | |
| 43 | BRAFi | 120 | | |
| 44 | BRAFi | 60 | | |
| 46 | BRAFi | 60 | MDM2i | 3000 |
| 47 | BRAFi | 60 | JAKi | 20 |
| 48 | BRAFi | 60 | PKCi | 3500 |
| 49 | BRAFi | 60 | SRCi | 2400 |
| 50 | BRAFi | 60 | STAT3i | 600 |
| 51 | BRAFi | 60 | mTORi | 0.3 |
| 52 | PKCi | 3500 | | |
| 53 | PKCi | 3500 | mTORi | 0.3 |
| 54 | PKCi | 7000 | | |
| 55 | CDK4i | 2,000 | | |
| 56 | CDK4i | 2,000 | MEKi | 1.5 |
| 57 | CDK4i | 2,000 | AKTi | 5000 |
| 58 | CDK4i | 2,000 | HDACi | 6000 |
| 59 | CDK4i | 2,000 | MDM2i | 3000 |
| 60 | CDK4i | 2,000 | JAKi | 20 |
| 61 | CDK4i | 2,000 | BRAFi | 60 |
| 62 | CDK4i | 2,000 | PKCi | 3500 |
| 63 | CDK4i | 2,000 | SRCi | 2400 |
| 64 | CDK4i | 2,000 | STAT3i | 600 |
| 65 | CDK4i | 2,000 | mTORi | 0.3 |
| 66 | CDK4i | 2,000 | PI3Ki | 600 |
| 67 | SRCi | 2,400 | | |
| 68 | SRCi | 2,400 | PKCi | 3500 |
| 69 | SRCi | 2,400 | mTORi | 0.3 |
| 70 | SRCi | 4,800 | | |
| 71 | STAT3i | 600 | | |
| 72 | STAT3i | 600 | HDACi | 6000 |
| 73 | STAT3i | 600 | MDM2i | 3000 |
| 74 | STAT3i | 600 | PKCi | 3500 |
| 75 | STAT3i | 600 | SRCi | 2400 |
| 76 | STAT3i | 600 | mTORi | 0.3 |
| 77 | STAT3i | 1200 | | |
| 78 | mTORi | 0.3 | | |
| 79 | mTORi | 0.6 | | |
| 80 | PI3Ki | 600 | | |
| 81 | PI3Ki | 600 | HDACi | 6000 |
| 82 | PI3Ki | 600 | MDM2i | 3000 |
| 83 | PI3Ki | 600 | JAKi | 20 |
| 84 | PI3Ki | 600 | BRAFi | 60 |
| 85 | PI3Ki | 600 | PKCi | 3500 |
| 86 | PI3Ki | 600 | SRCi | 2400 |
| 87 | PI3Ki | 600 | STAT3i | 600 |
| 88 | PI3Ki | 600 | mTORi | 0.3 |
| 89 | PI3Ki | 1,200 | | |

### Proteomic/Phenotypic Profiles

An important aspect of the data acquisition for network inference is combining the proteomic and cellular phenotypic data so that the resulting models quantitatively link the proteomic changes to global cellular responses. Towards this objective, the melanoma cells were profiled for their proteomic and phenotypic response under 89 perturbation conditions (FIG. 2B). Reverse phase protein arrays (RPPA) were used to collect drug response data for 138 proteomic (total and phospho levels) entities in all conditions. In parallel, the quantitative phenotypic response was measured in all conditions. The measured phenotypic responses are cell viability and cell cycle progression (e.g., G1/S/G2/G2M arrest phenotypes) as measured by a resazurin assay and flow cytometry, respectively (FIG. 2B).

### The Response Map

The high throughput phenotypic and proteomic profiles formed a response map of cells to systematic perturbations (FIGS. 2A-2B). The response map provided context specific experimental information on the associations between multiple system variables (e.g. proteomic entities) and outputs (e.g., phenotypes) under multiple conditions (e.g., perturbations). It was demonstrated through hierarchical clustering of the map that each targeted drug induces a distinct proteomic response and drugs targeting the same pathway led to overlapping responses in the SkMel133 cells (FIG. 2B). Through a clustering-driven pathway analysis, it was further shown that functionally related proteins (e.g., proteins on same or related pathways) respond similarly to targeted agents (FIGS. 2C, 9A and 9B).

A description of the response map as a set of uncoupled pairwise associations between proteomic and phenotypic entities is not sufficient for achieving systematic predictions. Therefore, quantitative models were built using the experimental response map. The models describe the coupled nature of the interactions between proteins and cellular events, as well as the nonlinear dynamics of cellular responses to drug perturbations.

### 2. Quantitative and Predictive Network Models of Signaling

### Network Models

Next, the experimental response map (FIG. 2) and the BP-based inference strategy (FIG. 1) were used to build quantitative network models of signaling in melanoma. In the models, each node quantified the relative response of a proteomic or phenotypic entity to perturbations with respect to the basal condition. Consequently, proteomic entities that did not respond to at least a single perturbation condition, did not contribute any constraints for inference. Such entities were eliminated from network modeling with a signal-to-noise analysis and included 82 of the 138 proteomic measurements in modeling. In addition to the proteomic nodes, the models contained 5 phenotypic nodes and 12 “activity nodes,” which couple the effect of the targeted perturbations to the other nodes in the network. In total, network models contained 99 nodes. The BP algorithm generates the probability distribution of edge strengths for every possible interaction between the nodes. The BP-guided decimation algorithm instantiates distinct network model configurations from the probability model.

The mathematical formulation of the BP-based network inference is suitable for both de novo modeling (e.g., modeling with no prior information) and modeling using prior information. In certain embodiments, prior information was used to infer models with higher accuracy and predictive power compared to de novo models. Using the PERA computational tool, a generic prior information model was derived from Reactome and NCI-Nature PID databases, which are stored in Pathway Commons. The prior information network contains 154 interactions spanning multiple pathways (FIG. 10).

FIG. 10 illustrates a prior model of signaling. The prior information model is extracted from Reactome and NCI PID using the PERA algorithm. The prior model served as a bias in BP-based network inference. Dashed arrows represent priors for activating edges, red arrows represent inhibitory edges and black arrows represent generic edges (e.g., activating or inhibitory).

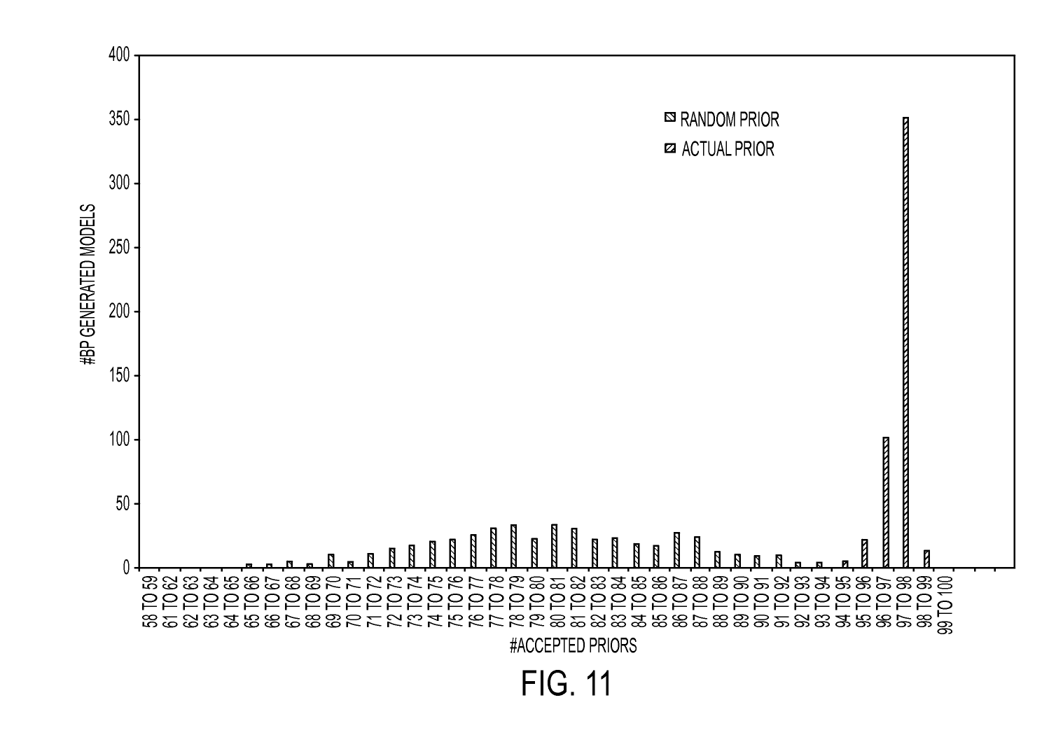

In turn, a prior prize term was added to the error model to restrain the search space by favoring the interactions in the prior model. It is important that the prior information does not overly restrain the inferred models and the algorithm can reject incorrect priors. To address this problem, network models were inferred using the pathway driven and randomly generated prior restraints. The statistical comparison of the networks inferred with actual (e.g., reported in databases) and random prior models indicates that the inference algorithm rejects significantly higher number of prior interactions when randomly generated priors are used for modeling (FIG. 11).

FIG. 11 shows whether the database driven prior model conforms to the experimental data and if prior information does not overly restrain the inferred models. For this purpose, network models using the pathway driven and randomly generated prior restraints in BP-based modeling were constructed and compared. 500 unique, randomly generated prior restraints each containing 154 interactions, equal to the number of interactions in the pathway driven prior model were tested. For model construction, the probability distribution of edge values (P(Wᵢⱼ)) was first computed for each possible interaction. Next, the models were constructed by assigning the edge value (Wᵢⱼ) for which BP-generated P(Wᵢⱼ) is maximum. The models were compared and generated with different prior models for their prior scores (e.g., number of edges, which were accepted by the algorithm and also contained in the prior model). The models generated using the database driven priors had significantly higher prior scores (ps) compared to randomly generated models (p<0.05 Student's t-test for H0: μₚₛ^{random}=μₚₛ^{database-driven}). Note that, 500 parallel BP runs were performed with identical database driven prior models and experimental data. A low degree of variation was observed in the prior scores for the models with database driven priors. This is indicative of some solution degeneracy in the models inferred with database driven priors. In predictive network modeling, the solution degeneracy was accounted for by re-computing the P(Wᵢⱼ) at the beginning of each BP-guided decimation step and ensuring that the final solution ensemble contains models that sample every possible degenerate solution.

Finally, the experimental data and prior information were integrated to generate 4000 distinct and executable model solutions with low errors using the BP-based strategy.

FIG. 5 depicts a comparison of random versus actual prior information. The database driven prior model was tested to determine whether it conforms to the experimental data, and prior information does not overly restrain the inferred models. For this purpose, network models were constructed and compared using the pathway driven and randomly generated prior restraints in BP-based modeling. 500 unique, randomly generated prior restraints each containing 154 interactions, equal to the number of interactions in the pathway driven prior model, were tested. For model construction, the probability distribution of edge values (P(Wᵢⱼ)) was computed for each possible interaction. Next, the models were constructed by assigning the edge value (Wᵢⱼ) for which BP-generated P(Wᵢⱼ) is maximum. The models generated were compared with different prior models for their prior scores (e.g. number of edges which were accepted by the algorithm and also contained in the prior model). The models generated using the database driven priors had significantly higher prior scores (ps) compared to randomly generated models (p<0.05 Student's t-test for H0: μₚₛ^{random}=μₚₛ^{database-driven}). 500 parallel BP runs were performed with identical database driven prior models and experimental data. A low degree of variation in the prior scores was observed for the models with database driven priors. This is indicative of some solution degeneracy in the models inferred with database driven priors. In predictive network modeling, the solution degeneracy was accounted for by re-computing the P(Wᵢⱼ) at the beginning of each BP-guided decimation step and ensuring that the final solution ensemble contains models that sample every possible degenerate solution.

### 3. Use of Prior Information Improves the Predictive Power of Models

Cross Validations with and without Prior Information

Tests were performed to address the question of whether BP-derived models have predictive power and whether use of prior information introduces further improvement. To assess the predictive power of the network models (e.g., predicting the response to untested perturbations), a leave-k-out cross validation was performed. In two separate validation calculations, the response profile to every combination of either RAFi or AKTi was withheld (leave-11-out cross validation). This procedure created a partial training dataset that contains response to combinations of 11 drugs and 2 different doses of a single drug totaling to 78 unique conditions (Table 3).

First, both de novo (e.g., without any prior information) and prior information guided network models were conducted with each partial dataset. Next, the response in withheld conditions was predicted by executing the models with in silico perturbations that correspond to the withheld experimental conditions. Finally, the hidden and predicted response data from models generated de novo or with prior information were compared.

Restraining Inference with Prior Information Improves Predictive Power of Models

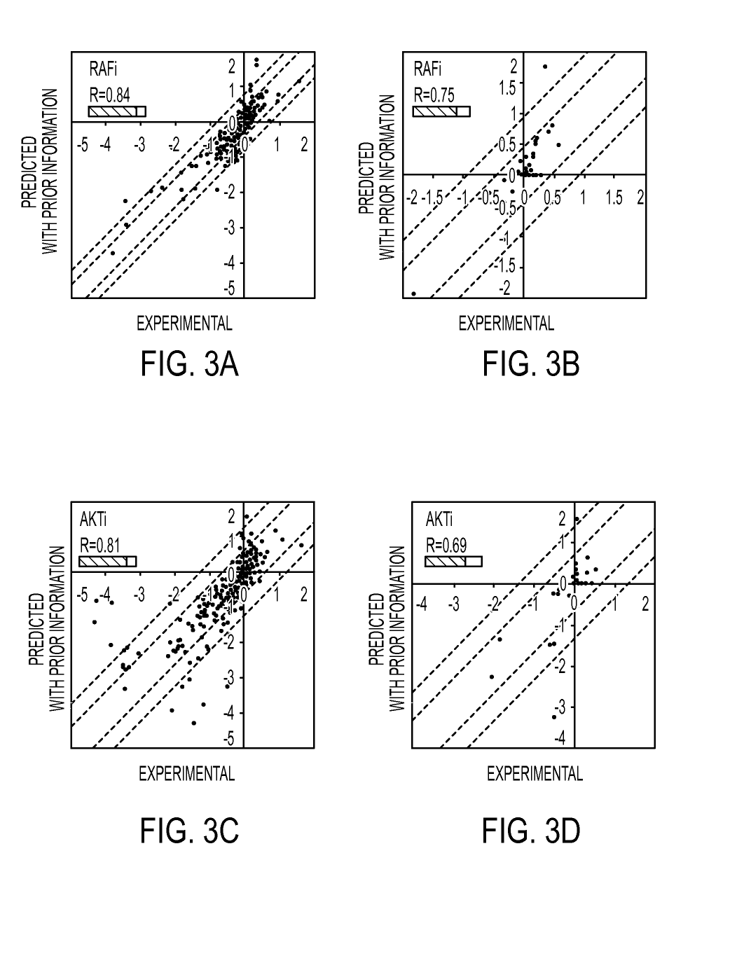

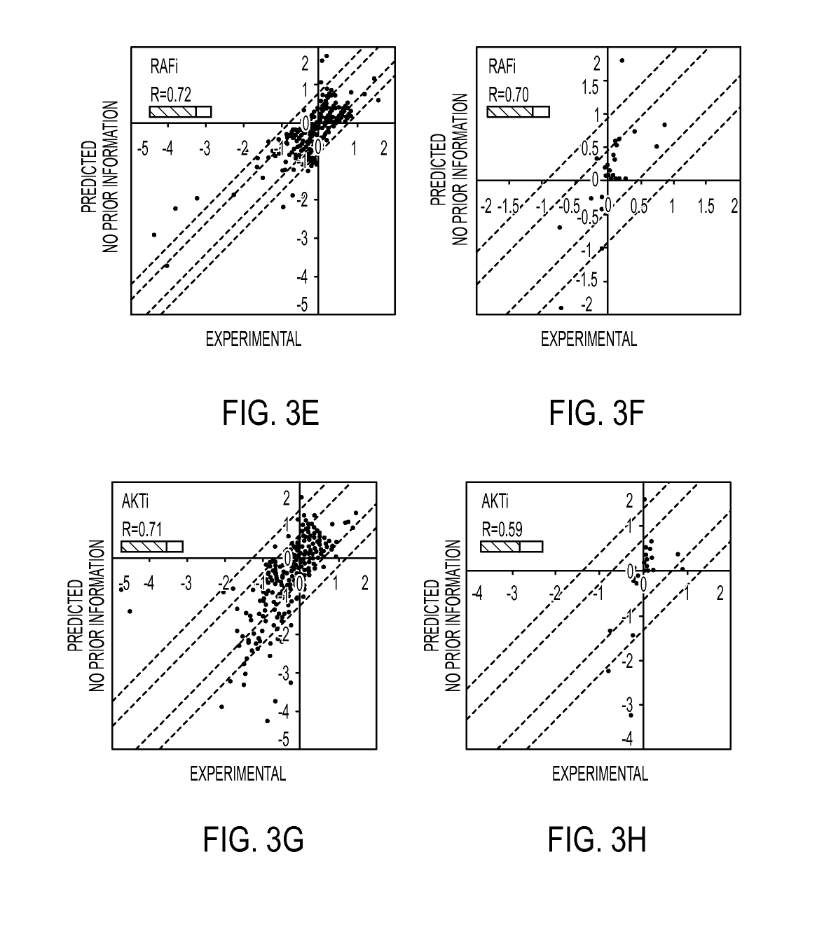

FIGS. 3A-3H illustrate how a use of prior information increases the predictive power of models. To test the predictive power of network models, a leave-11-out cross validation test was performed. Using the BP-guided decimation algorithm, 4000 network model solutions were inferred in the presence and absence of prior information using the partial response data. Resulting models were executed with in silico perturbations to predict the withheld conditions. Each experimental data point represented the read-outs from RPPA and phenotype measurements under the corresponding perturbation conditions. Each predicted data point was obtained by averaging results from simulations with in silico perturbations over 4000 model solutions. The experimental and predicted profiles were compared to demonstrate the power of network models to predict response to combinatorial drug perturbations. In all conditions, network inference with prior information led to a higher cumulative correlation coefficient (R) and significantly improved prediction quality (RAFi p=1×10-3, AKTi p=5.7×10-3, unpaired t-test H0: ΔXwith_prior=ΔXw/o_prior, ΔX=|Xexp−Xpred|) between experimental and predicted responses (FIGS. 3A-3D). Prior information was used for network inference, as shown in FIGS. 3E-3H, and no prior information was also used for network inference, as shown in FIGS. 3A, 3B, 3E, and 3F. Response to RAFi+{Di} was withheld from the training set and the withheld response was predicted (FIGS. 3C, 3D, 3G, 3H). Response to AKTi+{Di} was withheld from the training set and the withheld response was predicted (FIGS. 3A, 3C, 3E, 3G). All responses (phenotypic+proteomic) were plotted, and, in some embodiments, only phenotypic responses were plotted (FIGS. 3B, 3D, 3F, 3H). {Di} denotes set of all drug perturbations combined with drug of interest.

The comparison between the predicted and the withheld experimental profiles suggests that the de novo network models have considerable predictive power and the use of prior information in modeling in general introduces significant improvement in the prediction quality (FIGS. 3A-3H). Use of prior information increased the cumulative correlation coefficient between predicted and experimental response data from 0.72 to 0.84 and from 0.71 to 0.81 for RAFi and AKTi respectively. Without being bound by theory, the demonstrated predictive power of the models suggest that the models are suitable for systematically predicting response to perturbation combinations not sampled in the training set and generating novel hypotheses related to drug response in cancer cells.

### 4. Network Models Identify Context Dependent Oncogenic Signaling in Melanoma

### Network Modeling and the Average Model

Next, the quantitative network models were generated with the complete experimental response profile and the prior information interaction biases to investigate oncogenic signaling in melanoma. The resulting network models resembled conventional pathway representations facilitating their comparison with the biological literature, but the interaction edges did not necessarily represent physical interactions between connected nodes. Analysis of the ensemble of network model solutions revealed that a set of strong interactions is shared by a majority of the inferred low-error network models.

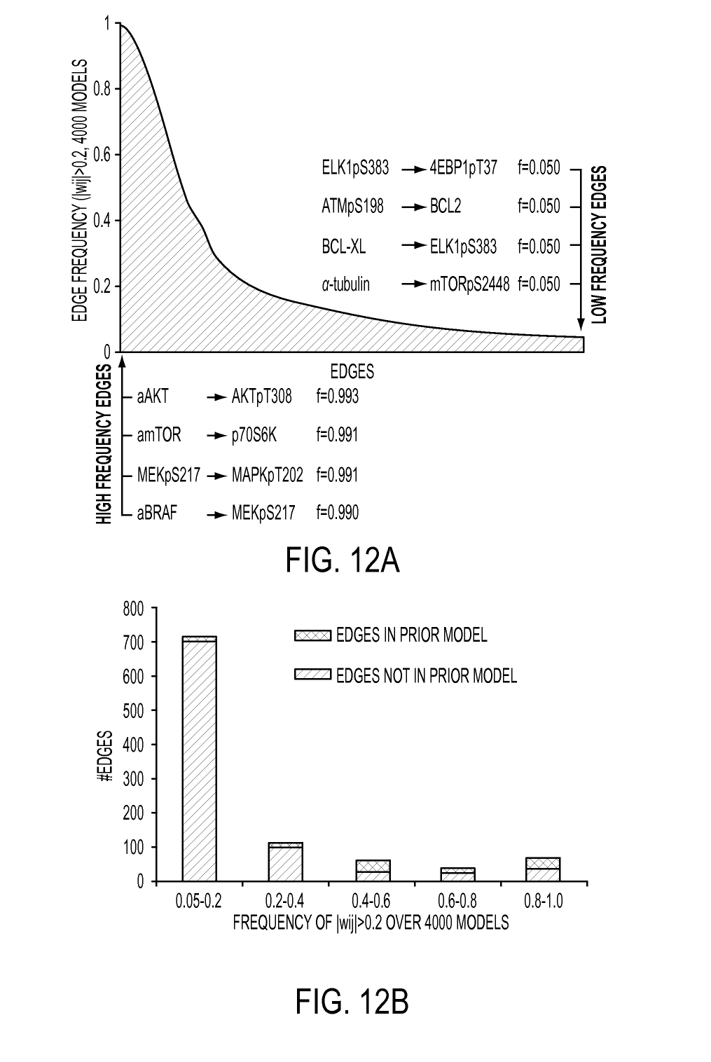

On the other hand, some interactions had non-zero edge strength (Wᵢⱼ) values only in a fraction of the models. FIGS. 12A-12B illustrate analysis of edge distribution in models. More specifically, FIGS. 12A and 12B illustrate a distribution of edges in the solution ensemble. In order to model cellular signaling and predict response, 4000 signaling models were computed and generated a solution ensemble. In FIG. 12A, edge frequencies (f(wᵢⱼ) are in the solution ensemble. The y-axis represents the frequency values for nonzero edge strengths (f(|wᵢⱼ|>0.2)) were in the ensemble. The X-axis represents the interactions ordered by their f(|wᵢⱼ|>0.2). The long tail that corresponds to the edges with frequencies less than 0.05 is not displayed. A set of well reported interactions such as the coupling of AKT activity node to phospho-AKT and phospho-MEK to phosho-MAPK (ERK) were inferred with |wᵢⱼ|>0.2 in >99% of the model solutions (left). Few examples of the rarely captured signaling interactions are given (right).

In FIG. 12B, the frequency distribution of nonzero edges (|wᵢⱼ|>0.2) has a bimodal character. A set of interactions with nonzero edge values was shared by nearly all of the inferred network models (0.8<f(|wᵢⱼ|>0.2)<1.0). Such frequent edges formed a stable network skeleton shared by majority of solutions. On the other hand, a set of possible interactions had non-zero edge strength (wᵢⱼ) values in around 50% of the solutions. Such interactions varied among different model solutions and possibly accounted for the variations in system response predicted with different model solutions. The interactions in the prior model were particularly enriched in these two groups. A large fraction of potential interactions had very low (e.g., 0.05<f(|wᵢⱼ|>0.2)<0.20) or near zero (e.g., f(|wᵢⱼ|>0.2)<0.05) frequencies in the model ensemble.

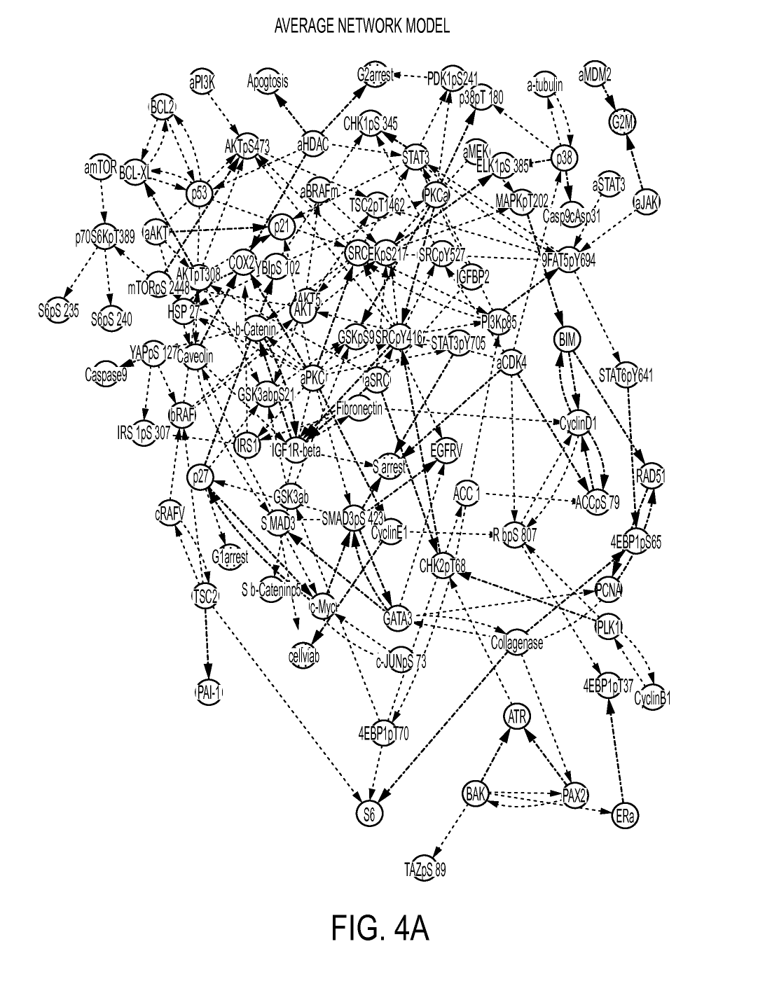

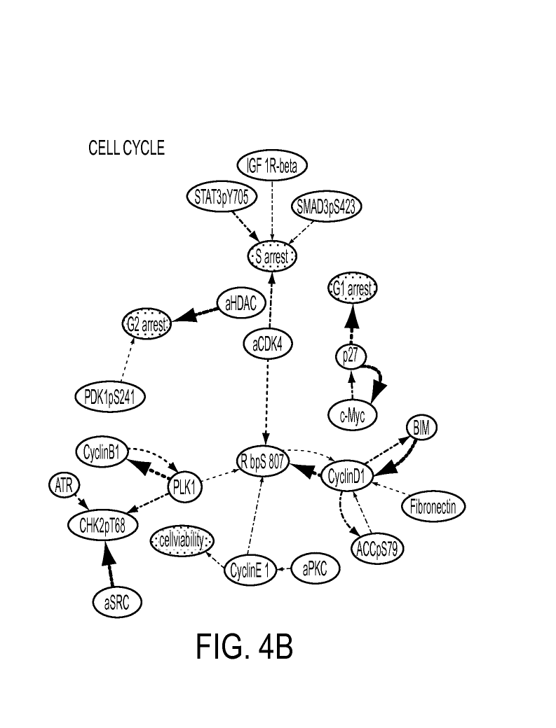

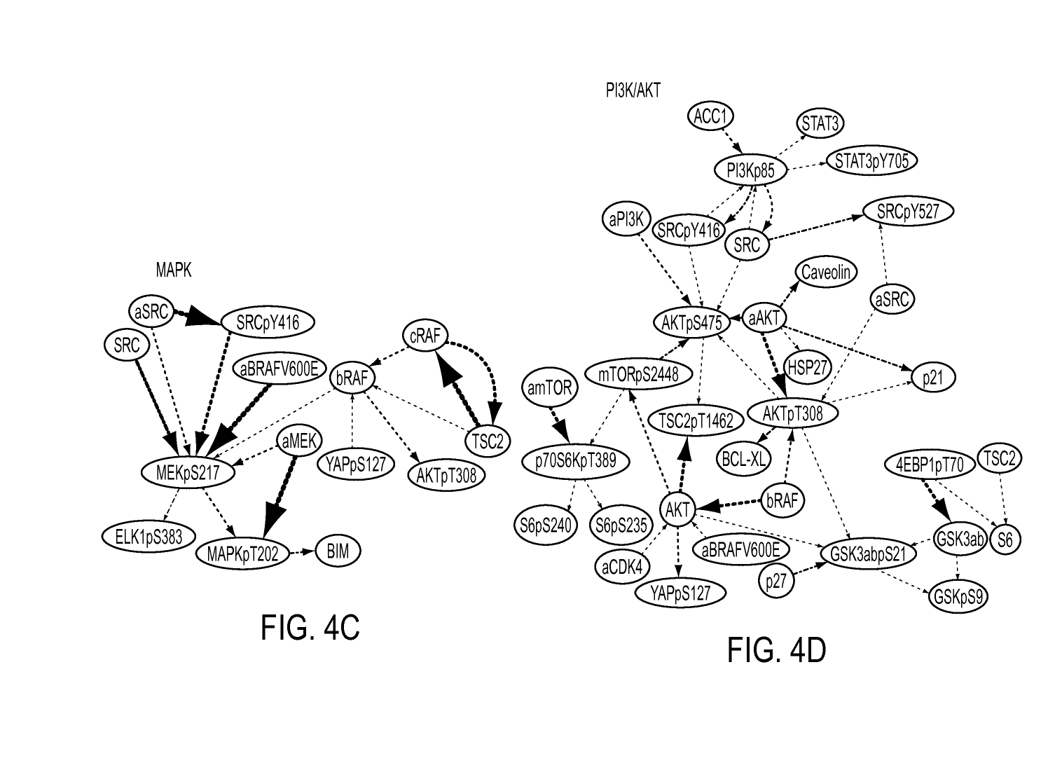

This variation correctly and usefully reflects uncertainty and incompleteness in the data, as well as nearly degenerate (with respect to the error function) alternative network models. As a first step of detailed analysis and for the purpose of intuitive interpretation, an average network model (for example, as shown in FIG. 4A) was computed, which was obtained by averaging the interaction strength (Wᵢⱼ) for each node pair ij over all individual model solutions. This average network model highlights the interactions (Wᵢⱼ) with high interaction strengths if shared by the majority of the distinct solutions. Although the average model cannot be simulated to predict system response, it is particularly useful for qualitative analysis of the inferred signaling interactions.

### Global Analysis of Average Models

The average network model provided a detailed overview of the signaling events in melanoma cells (FIG. 3A). The average model contained 203 unique interactions (127 activating and 76 inhibitory interactions) between 99 signaling entities. 89 of the 154 interactions in the prior model conformed to the experimental data and, therefore, were accepted in a majority of the model solutions by the inference algorithm and included in the average model. Given that the average model covered interactions from multiple signaling pathways and was more complex than most pathway diagrams, even the qualitative analysis of the model was highly challenging.

### Network Models Capture Known Signaling Pathways

That is, in order to simplify the analysis of the average model solution, the global network diagram in FIG. 4A was fragmented into sub-networks. Each subnetwork is a simplified representation of the signaling events in canonical pathways such as those that fall into MAPK, PI3K/AKT and cell cycle pathways (FIGS. 4B-4D). The sub-network diagrams indicate that models recapitulate many known interactions in pathways, which are important in melanoma tumorigenesis (e.g., PI3K/AKT and MAPK) and nominate previously unidentified interactions (FIGS. 4A-4D). It is, however, not possible to predict the cellular response to untested drug perturbations through qualitative analysis of the inferred interactions. Quantitative simulations were used with in silico perturbations to both decode the signaling mechanisms and, more importantly, systematically predict cellular response to combinatorial drug perturbations.

In detail, FIGS. 4A-4D illustrate inferred network models that capture oncogenic signaling pathways in melanoma. In FIG. 4A, the average network model contains proteomic (white) and phenotypic nodes (dashed) and the average signaling interactions (<Wij>>0.2) over the model solutions. The edges between the BRAF, CRAF, TSC2 and AKTpT308 represent the cross-pathway interactions between the MAPK and PI3K/AKT pathways. In FIG. 4B, cell cycle signaling sub-network contained well-known interactions between the Cyclins, CDKs and other associated molecules (e.g., p27/Kip1). RbpS807 and CyclinD1 are the hub nodes in the sub-network and connect multiple signaling entities. In FIG. 4C, a MAPK sub-network is shown. MEKpS217 is the critical hub in this pathway that MEK phosphorylation links upstream BRAF and SRC to downstream effectors such as MAPK phosphorylation. In FIG. 4D, the PI3K/AKT sub-network is shown, the SRC nodes (e.g., phosphorylation, total level, activity) are upstream of PI3K and AKT (total level, AKTpS473 and AKTp308), and the AKT nodes are the major hubs. Downstream of AKT, the pathway branches to mTOR, p70S6k and S6 phosphorylation cascade and the GSK3 phosphorylation events. A negative edge originating from mTORpS2448 and acting on AKTpS473 presumably captures the well-defined negative feedback loop in the AKT pathway. Nodes tagged with “a” (e.g., aBRAF) are activity nodes.

### 5. Combinatorial in Silico Perturbations Generate an Expanded Proteomic/Phenotypic Response Map

Model Execution with in Silico Perturbations

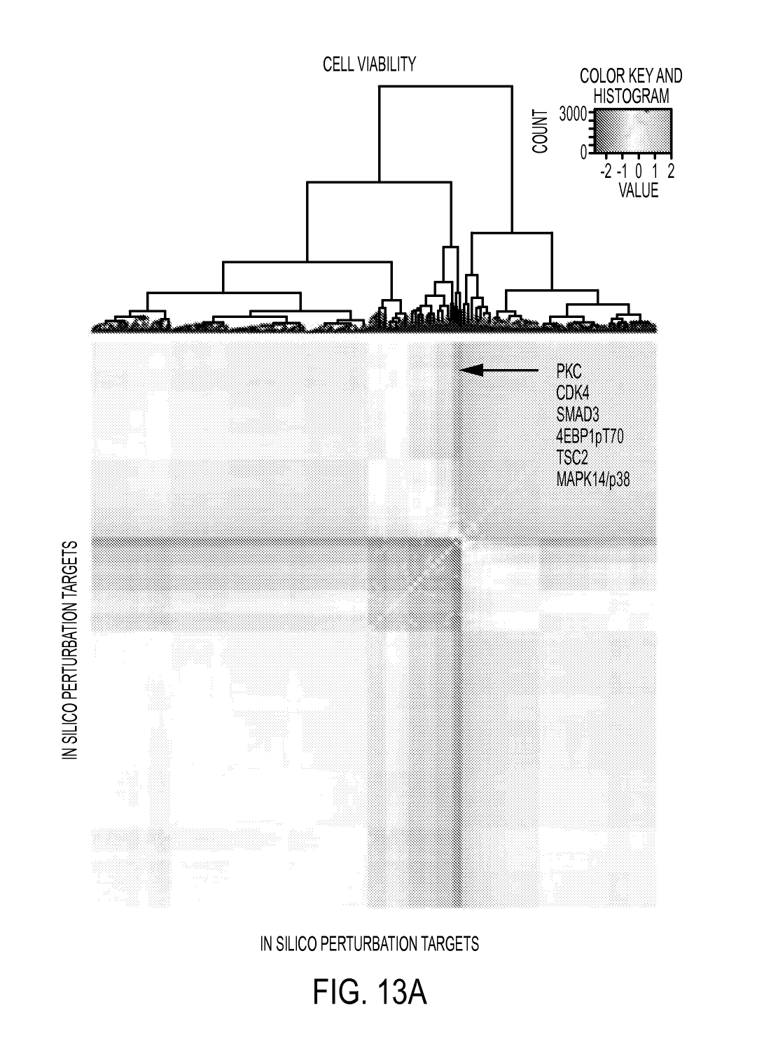

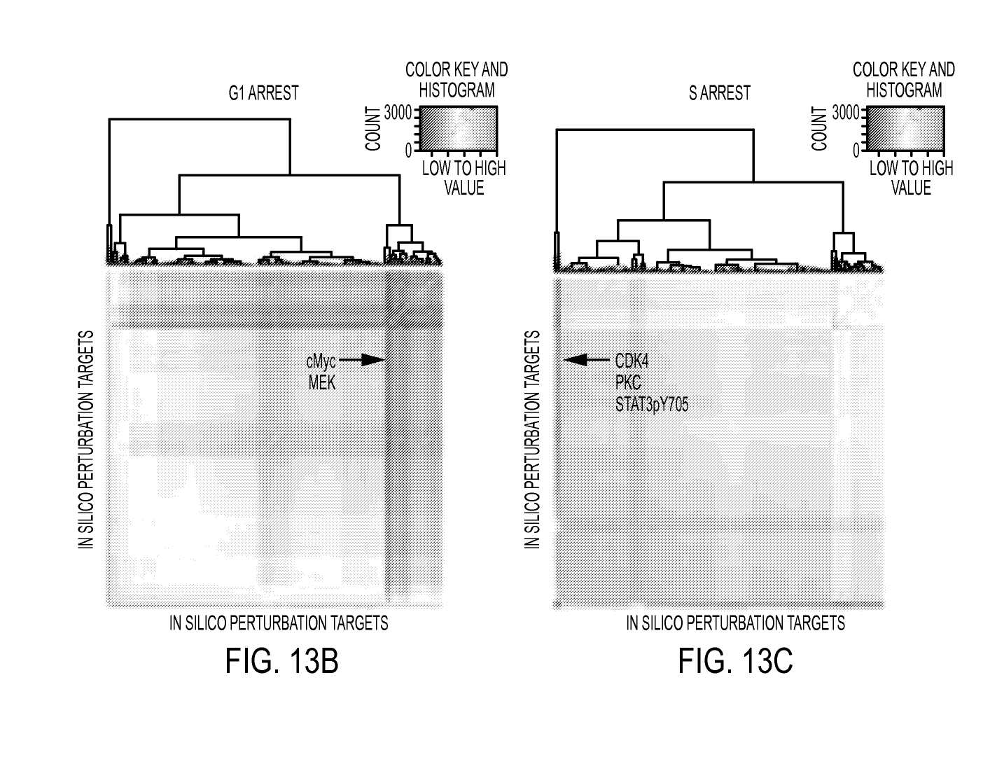

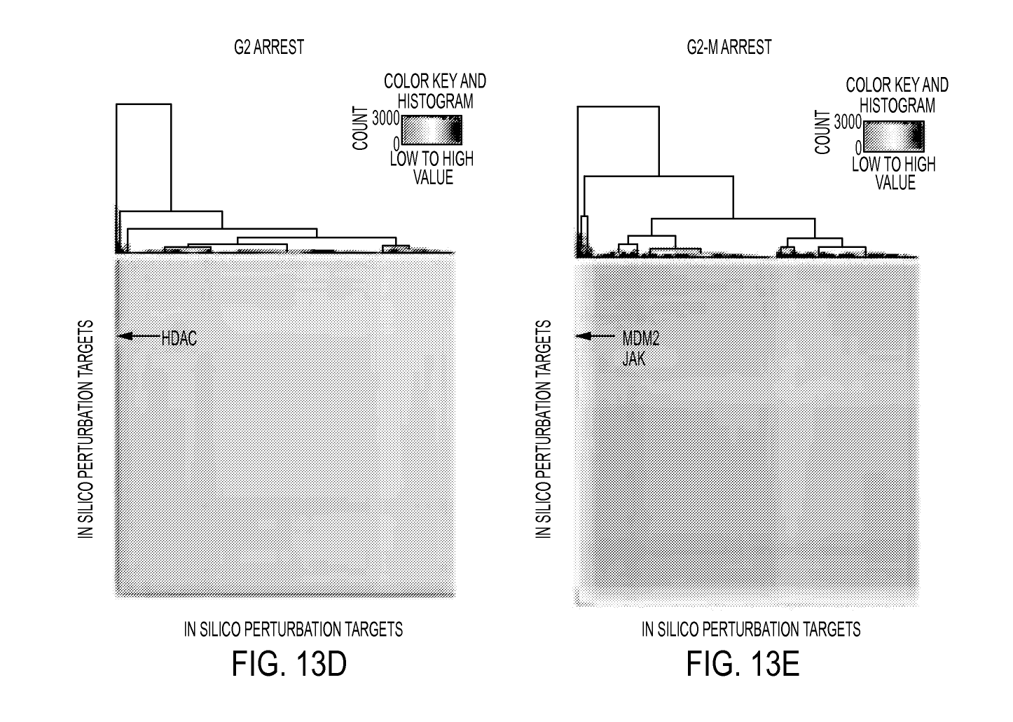

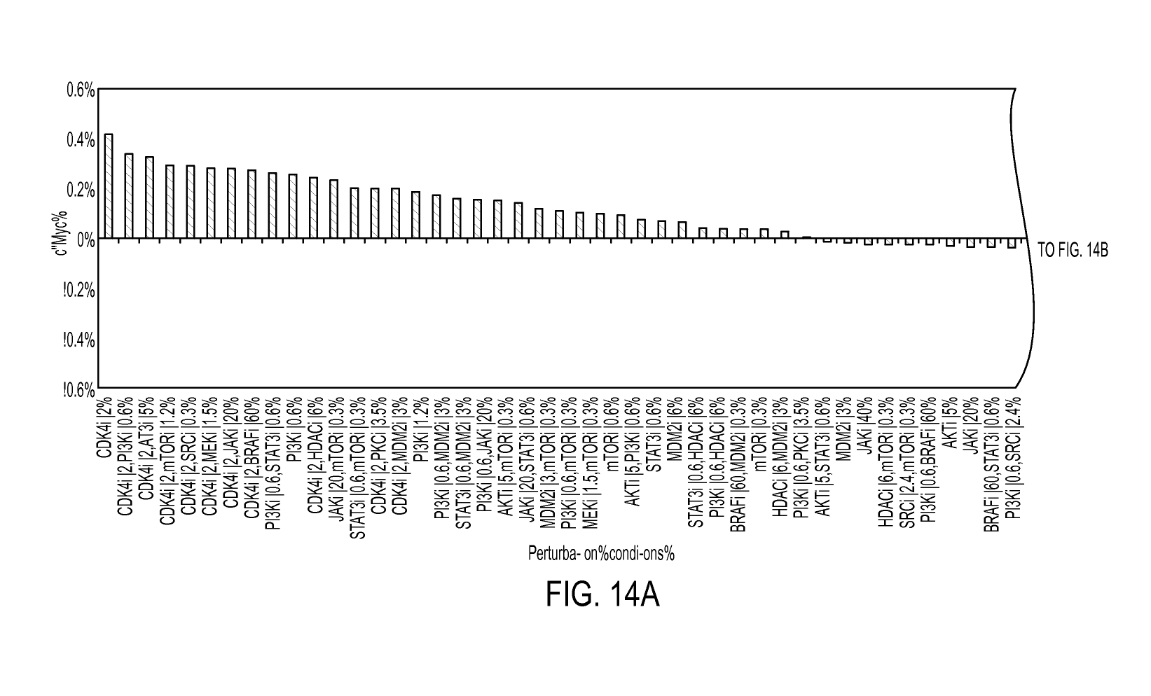

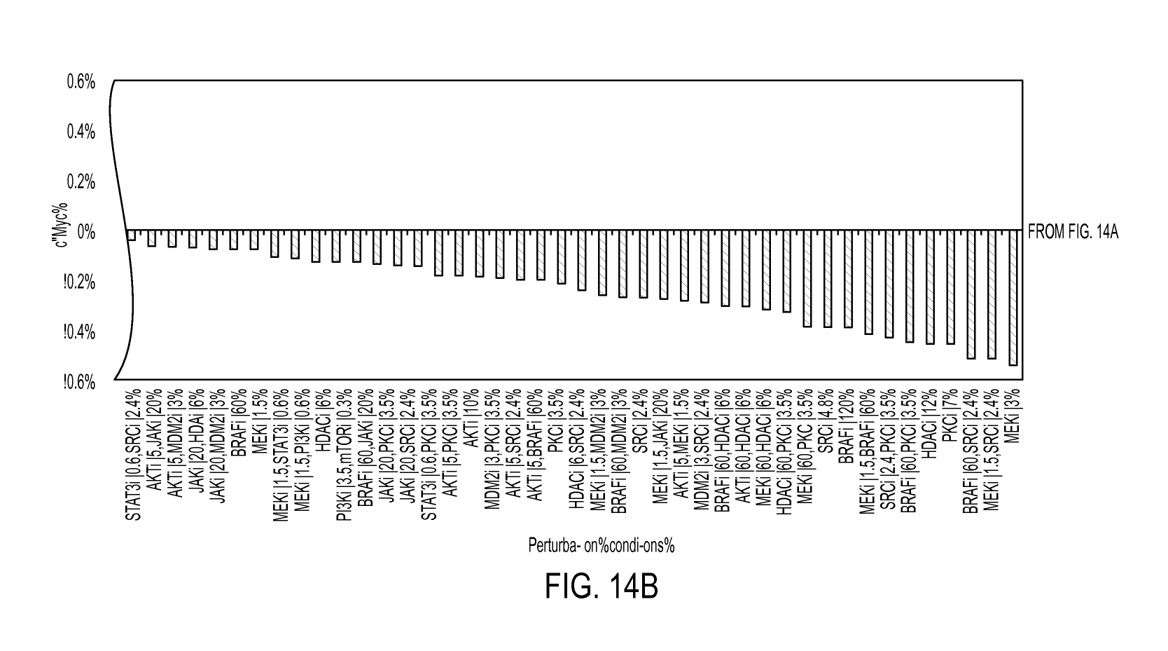

FIGS. 5A-5H illustrate how simulations with in silico perturbations provide predictions on system response to novel perturbations. FIG. 5A shows the schematic description of network simulations. The system response to paired perturbations was predicted by executing the ODE-based network models with in silico perturbations. In the ODE based models, Wij represented the interaction strengths and were inferred with the BP-based modeling strategy. The in silico perturbations were applied as real valued uim vectors. The time derivative and final concentration of any predicted node was a function of the model parameters, the perturbations and the values of all the direct and indirect upstream nodes in the models. FIG. 5B shows how the model equations were executed until all model variables (protein and phenotype responses) reached steady state. The predicted response values were the averages of simulated values at steady state over multiple distinct model solutions. In FIG. 5C, the simulations expanded the response map by three orders of magnitude and generate testable hypotheses. FIGS. 5D-5H show the top ten most effective single-agent in silico perturbations for each phenotype. The listed proteomic entities for each phenotype are potential drug targets for slowing down the growth of melanoma cell. The error bars represent the ±SEM over the simulations of all model solutions. FIGS. 13A-13E, and FIG. 14 illustrate prediction heatmaps for phenotypes.

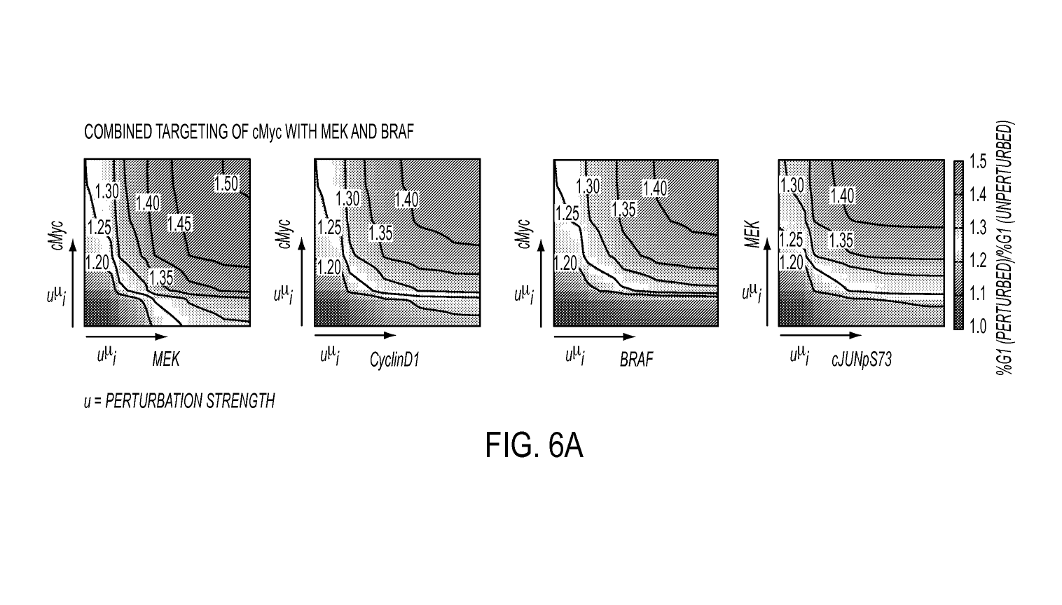

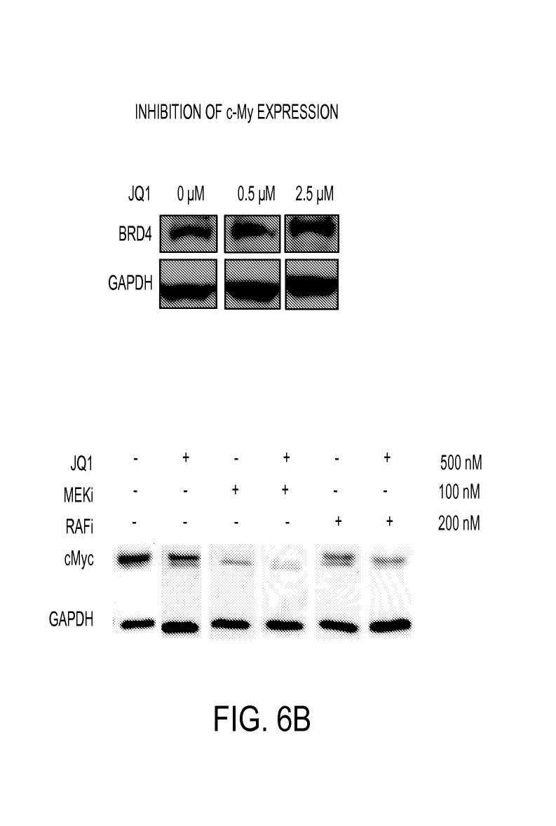

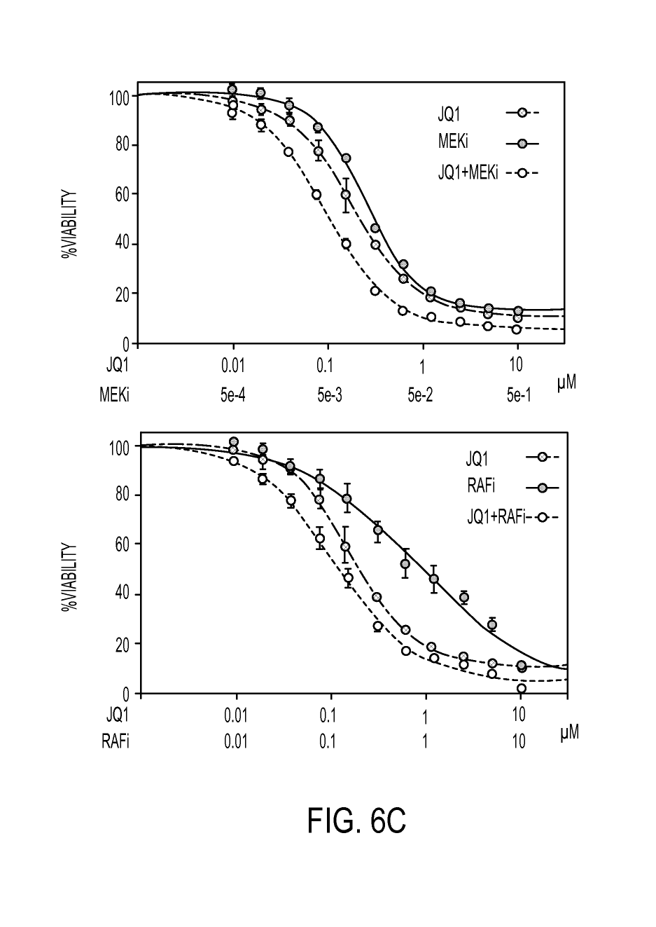

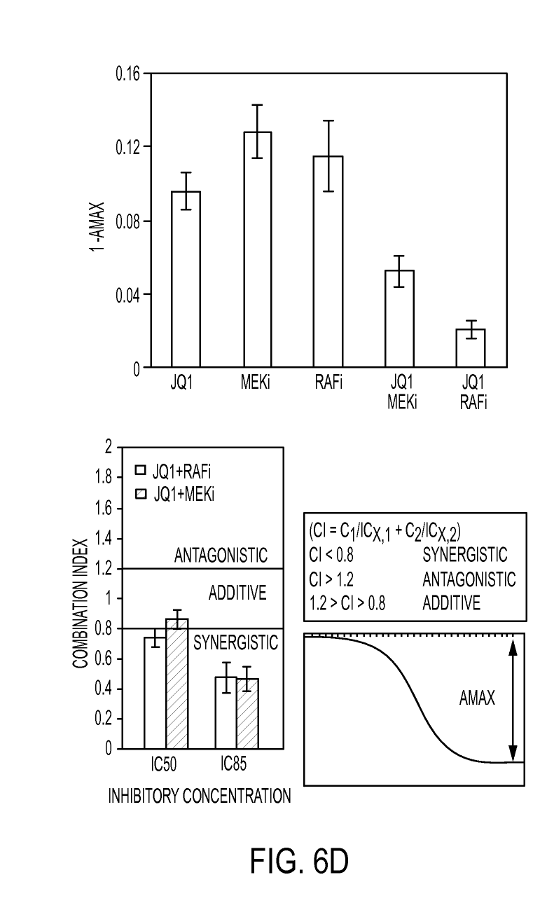

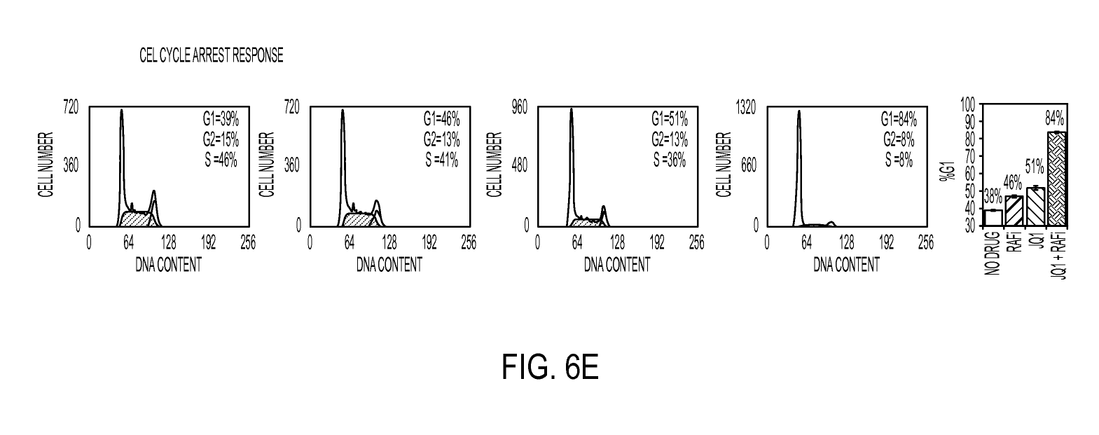

FIGS. 13A-13E depict a prediction of phenotypic responses to in silico, combinatorial perturbations. The cell viability and cell cycle arrest (G1, S, G2 and G2M) was computationally predicted in response to combinatorial perturbations of all proteomic nodes in network models (Table 1; FIGS. 5A-5H, and 6A-6E). In silico perturbations included paired combinations of 94 perturbations in 5 different concentrations (IC0-20-40-60-80 with respect to the basal level of a particular node) leading to more than 70000 unique perturbation conditions. Each in silico perturbation was applied to 4000 inferred network models. This led to a simulation of ˜28 million unique conditions for 99 different readouts, a throughput level inaccessible by experimental screening methods. The heat maps displayed the phenotypic response landscape to combinatorial perturbations. Each data point on the heat maps was the response to a perturbation averaged over predictions from 4000 network models. FIG. 13A depicts a cell viability response to perturbations. The desired response from a perturbation was reduction in cell viability (Darker Grey: decreased cell viability; Lighter grey: increased cell viability). FIGS. 13B-13E depict a similar result as shown in FIG. 13A but for cell cycle progression phenotypes. For cell cycle phenotypes, the desired response was an increase in the cell cycle arrest (Darker grey: increased cell cycle arrest; Lighter grey: decreased cell cycle arrest).

FIG. 14 depicts changes in c-Myc levels in response to perturbations in Skmel133 cells as measured in RPPA experiments. Each data point was log normalized with respect to the c-Myc level in unperturbed condition. c-Myc level was highest in the presence of CDK4i. Various drug combinations that included STAT3 and mTOR inhibitors follow CDK4i combinations. cMyc level was lowest when cells were perturbed with MEK, BRAF, SRC, PKC and HDAC inhibitors, respectively. Each data-point is the average of RPPA readouts from three replicates.

Because of their ODE-based mathematical descriptions, the models can be executed to predict cellular responses to novel perturbations. The systematic predictions go beyond the analysis of few particular edges in the system and capture the collective signaling mechanisms of response to drugs from the modeled pathways. The parameterized model ODEs (Equation 1) was executed with in silico perturbations acting on node (i) as a real numbered u(i) value until all the system variables (e.g., node values, {xᵢ,}) reach to steady state (FIG. 5A-B). The simulations expand the size of the response map by three orders of magnitude from a few thousand experimental response data to millions of predicted responses (FIG. 5C).

### Prediction of Phenotypic Responses

The simulations enabled identification of effective perturbation combinations that cannot be trivially deduced from the experimental data (Table 4; FIGS. 5D-H, 13 and 14). Once the predicted response profiles were obtained, specific perturbations that may induce desired phenotypic changes were sought. Predictions suggested that c-Myc links multiple pathways such as MAPK and PI3K/AKT to cell cycle arrest (Table 4), consistent with the vast literature on c-Myc's role in genesis of many cancers. A thorough analysis of all the predicted responses suggests that melanoma cells are arrested in G1-phase when c-Myc is targeted particularly in combination with BRAF, MEK and CyclinD1 molecules (FIG. 6A). As neither c-Myc nor its direct regulators were inhibited in the perturbation experiments, the predictions from the models were nontrivial. Table 4 below shows phenotypic nodes and predicted responses from simulations with in silico perturbations.

TABLE 4

| | Immediate | | |
|---|---|---|---|
| | upstream nodes | Predicted | Predicted |
| | (<wᵢⱼ> in average | primary | combination |
| Phenotype | model) | target* | partners |
| Cell | SMAD3 (0.49) | aPKC | SMAD3, 4EBP1pT70, TSC2, |
| viability | CyclinE1 (−0.28) | aCDK4 | MAPK14/p38 |
| G1-arrest | p27/Kip1 (0.83) | c-Myc | Cell cycle (CyclinB1, |
| | PDK1pS241 | aMEK | RbpS807, |
| | (0.20) | | CyclinD1, PLK1), |
| | | | MAPK pathway (MAPKpT202, |
| | | | MEKpS217, BRAF, aBRAF), |
| | | | AKT, AKTpT308 , RAD51, |
| | | | p38/MAPK14, aSRC, YBIpS102, |
| | | | cJUNpS73, SMAD3 |
| G2-arrest | aHDAC (−0.77) | aHDAC | Generic response from all nodes |
| S-arrest | STAT3pY705 | aCDK4 | aPKC, SMAD3pS423, |
| | (−0.66) | | STAT3pS705, IGFBP2, |
| | IGF1Rβ (−0.20) | | CyclinB1, IGF1Rβ, Fibronectin |
| | SMAD3pS423 | aPKC | STAT3pS705 |
| | (−0.23) | | |
| | aCDK4 (−0.49) | | |
| G2M | aJAK (−0.24) | aMDM2 | aJAK, aSTAT3, BRAF, |
| | aMDM2 (−0.38) | | 4EBPpT70 |

A node is defined as a primary target when substantial phenotypic change is predicted in response to perturbation of the node alone. The phenotypic response is further increased when the primary targets are perturbed in combination with a set of other nodes (e.g., the combination partners).

### 6. Co-Targeting c-Myc with MEK or BRAF is Synergistic in Melanoma Cells

FIGS. 6A-6E illustrate the combined targeting of c-Myc with MEK and BRAF leads to synergistic response in melanoma cells. FIG. 6A shows the isobolograms of predicted G1-response to combined targeting of c-Myc with MEK, BRAF, CyclinD1 and pJUNpS73. The leftward shift of isocurves implies synergistic interactions between the applied perturbations. u denotes strength of in silico perturbations. In FIG. 6B, the top panel shows western blots show the level of BRD4 24 hours after JQ1 treatment. The BRD4 level is unchanged in response to JQ1 treatment. The bottom panel of FIG. 6B shows western blots show the expression levels of c-Myc in response to JQ1, MEKi, RAFi and their combinations 24 hours after drug treatment. C-Myc expression is targeted with BRD4 inhibitor, JQ1. FIG. 6C top panel shows the cell viability drug dose-response curves for MEKi and JQ1. Cell viability is measured using the resazurin assay. FIG. 6C bottom panel shows the cell viability drug dose-response curves for RAFi and JQ1. FIG. 6D shows the synergistic interactions between JQ1 and RAFi/MEKi. The combination index (CI) calculated using the drug dose response curves quantifies the drug interactions between JQ1, MEKi and RAFi (left panel). CI for a given level of inhibition is a measure of the fractional shift between the combination doses (C1 and C2) and the single agent's inhibitory concentration (Cx,1, Cx,2). It is formulated as CI=(C1/Cx,1)+=(C2/Cx,2). Amax is the response of melanoma cells to JQ1, MEKi and RAFi at highest doses applied (right panel). 1-Amax is the fraction of cells alive in response to highest drug dose normalized with respect to the non-drug treated condition. FIG. 6E shows the cell cycle progression phenotype in response to JQ1. 86% of cells are in G1 state 48 hours after JQ1 treatment, while 42% of the cells are in G1 state before drug treatment (error bars: ±SEM in 3 biological replicates).

The key predictions based on the network modeling were experimentally tested. It was predicted that growth of melanoma cells is impaired when c-Myc is targeted alone and in combination with other proteins, particularly BRAF, MEK and CyclinD1 (FIG. 6A). In order to target c-Myc, melanoma cells were treated with the BET bromodomain inhibitor, JQ1 as a single agent and in combination with MEKi and RAFi. In multiple tumor types, JQ1 directly targets the bromodomain protein BRD4, which marks select genes including c-Myc on mitotic chromatin. Inhibition of BRD4 with JQ1 downregulates the c-Myc mRNA transcription and leads to G1 cell cycle arrest in multiple tumor types.

First, it was postulated whether c-Myc levels in SkMel133 cells could be targeted using JQ1. As measured by western blot experiments, c-Myc expression is reduced in response to JQ1 alone. c-Myc levels are further reduced when the cells are treated with combination of JQ1 and MEKi or RAFi (FIG. 6B). Therefore, total c-Myc levels are targeted using JQ1, either as a single agent or in combination with inhibitors of the RAF/MEK/ERK pathway.

Next, the effect of JQ1 on the viability of melanoma cells (SkMel133) was tested and it was demonstrated that the tested melanoma cells are sensitive to single agent JQ1 treatment (cell viability IC50=200 nM). The sensitivity of SkMel133 to JQ1 is comparable to the sensitivity of multiple myeloma and MYCN-amplified neuroblastoma samples and substantially higher than those of lung adenocarcinoma and MYCN-WT neuroblastoma cells.

The effect of combined targeting of c-Myc with MEK and BRAFV600E was tested in SkMel133 cells. Strikingly, when combined with JQ1 (120 nM), cell viability was reduced by 50% with 120 nM of RAFi, whereas the IC50 for single agent RAFi is >1 μM in RAFi resistant SkMel133 cells. Similarly, when combined with 5 nM MEK inhibitor, viability of SkMel133 cells was reduced by 50% with 100 nM of JQ1, an IC50 value, which is close to those of the most sensitive multiple myeloma cell lines. At higher doses (IC80), JQ1 is synergistic with both MEKi (CI₈₅=0.46) and RAFi (CI₈₅=0.47) in SkMel133 cells. At intermediate doses, JQ1 synergizes with RAFi (Combination index, CI₅₀=0.65) and has near additive interaction with the MEKi (CI₅₀=0.85) (FIG. 6D). Consistent with the observed synergy at high doses, both JQ1 combinations improve the maximal effect level (Aₘₐₓ, response to the drugs at highest doses) from 10% (JQ1), 13% (MEKi) and 12% (RAFi) to 5% (MEKi+JQ1) and 2% (RAFi+JQ1), leading to lower cell viability beyond the levels reached by treatment with any of the agents alone. The observed improvement in Aₘₐₓ is particularly important since a subpopulation of cancer cells usually resist treatment even at highest possible drug doses. Treatments with drug combinations, such as those tested here, can overcome or delay emergence of drug resistance if they can shrink the size of this resistant subpopulation (e.g., lead to improved Aₘₐₓ).

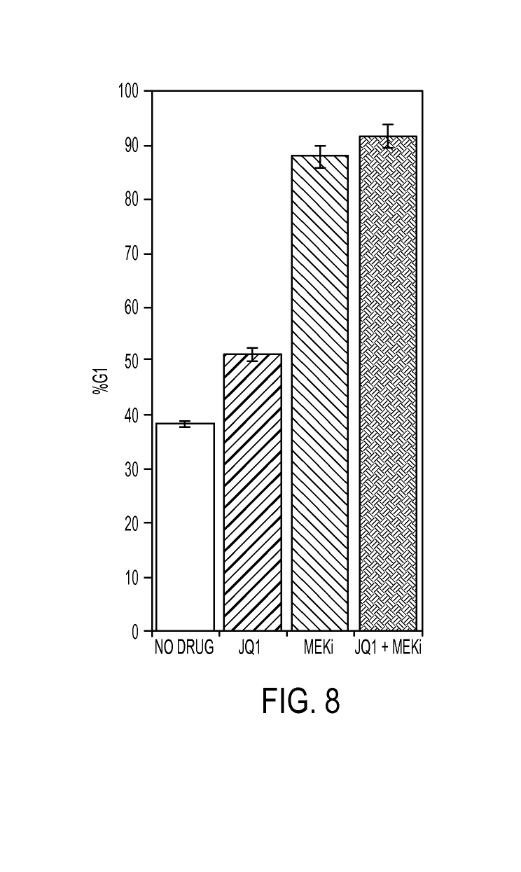

Finally, the cell cycle progression and apoptotic response of melanoma cells to JQ1 were measured. JQ1 (500 nM) induces a 50% increase in cells at G1-phase compared to the unperturbed cells (FIG. 8). FIG. 8 shows a modest increase in apoptosis is observed 48 hours after JQ1 treatment (11% as opposed to 3% in unperturbed cells). More specifically, FIG. 8 depicts cell cycle arrest response of melanoma cells to JQ1 and MEKi combination. 51% and 88% of cells are in G1 stage 24 hours after JQ1 and MEKi treatment, respectively. JQ1+MEKi combination has an increased effect on G1 cell cycle arrest (G1=92%). 39% of cells are in G1 when they are not treated with drugs (error bars in right panel: ±SE in 3 biological replicates).

### DISCUSSION

Predictive network models of signaling in melanoma cells were generated to systematically predict cellular response to untested drug perturbations. The modeling algorithm integrated information from high-throughput drug response profiles and pathway data from signaling databases. The scale and the predictive power of the models are beyond the reach of the currently available network modeling methods. Based on the predictions from models, it was found that co-targeting MEK or BRAF with c-Myc leads to synergistic responses to overcome RAF inhibitor resistance in melanoma cells. In various embodiments, this strategy may be applied for model-driven quantitative cell biology with diverse applications in many fields of biology.

### Cell Type Specificity

In network modeling, the experimental data provides the cell type specific constraints while the priors introduce a probabilistic bias for generic signaling information. Consequently, the network models are cell type specific and not only recapitulate known biology but also predict novel interactions. Moreover, the algorithm rejects a significant part of the interactions in the prior model. For example, the influences of Cyclin E1 and Cyclin D1 on RB phosphorylation are well known. The inferred models included the expected positive edge between Cyclin D1 and RBpS807, but not between Cyclin E1 and RBpS807. It is possible to identify the genomic features in Skmel133 cells that lead to such context specific interactions. The gene CDKN2A product, p16Ink4A directly inhibits Cyclin Dl/CDK4 as it participates in a G1 arrest checkpoint. On the other hand, the alternative CDKN2A gene product, p14ARF can inhibit Cyclin E1/CDK2 complex only indirectly through an MDM2/p53/p21Cif1 dependent pathway. CDKN2A gene products have no direct influence on Cyclin E1. In SkMel133 cells, the homozygous deletion in CDKN2A most likely leads to excessive Cyclin Dl/CDK4 catalytic activity, which may override the influence of Cyclin E1/CDK2 complex on RBpS807.

Co-Targeting c-Myc and BRAFV600 Signaling

Oncogenic alterations that decrease drug sensitivity may exist in combinations or emerge sequentially in a tumor. Therefore, it is likely that tumors can escape therapy through alternative routes. A counter strategy is identifying and targeting pivotal proteins on which multiple pathways converge. Through quantitative simulations, it was predicted that c-Myc couples multiple signaling pathways such as MAPK and AKT to cell cycle arrest in SkMel133 cells (Table 1). According to the predictions, co-targeting c-Myc with MEK, BRAF or CyclinD1 leads to the highest impairment in tumor growth. To test these predictions, c-Myc was targeted using the epigenetic drug JQ1, a BRD4 inhibitor that downregulates c-Myc transcription. Cells were treated with combinations of JQ1 and MEKi or RAFi. It was shown that both combinations lead to synergistic cell viability response with particularly improved outcomes in high doses (Aₘₐₓ). It is important that targeting c-Myc can be highly toxic, since c-Myc functions as a key transcription factor in almost all normal tissues and tumors. A potential solution to the toxicity problem is lowering the required drug doses by co-targeting c-Myc with synergistic partners that are altered only in tumor cells as described herein (e.g., BRAFV600E). At lower drug doses, less severe side effects in tissues are expected, which do not carry the altered gene products. It can be concluded that co-targeting c-Myc and BRAFV600E goes beyond single agent treatments in overcoming drug resistance and lowering drug toxicity in melanomas with the genomic context under development.

The model-based predictions provide comprehensive and testable hypotheses on complex regulatory mechanisms, drug response and development of novel therapies. The improvements in experimental data volume and signaling databases produce network models with even higher predictive power. Coupling of the cell line specific predictions to comprehensive genomic analyses guides extrapolation of the potential impact of the nominated combinations to tumors with similar genomic backgrounds. For example, genomics methods have been developed to classify tumors based on select oncogenic alterations and compare tumor and cell line samples. By integrating network models, genomics and pathway analysis, it is possible to generate whole cell models of signaling and drug response in mammalian cells with potential applications in personalized medicine.

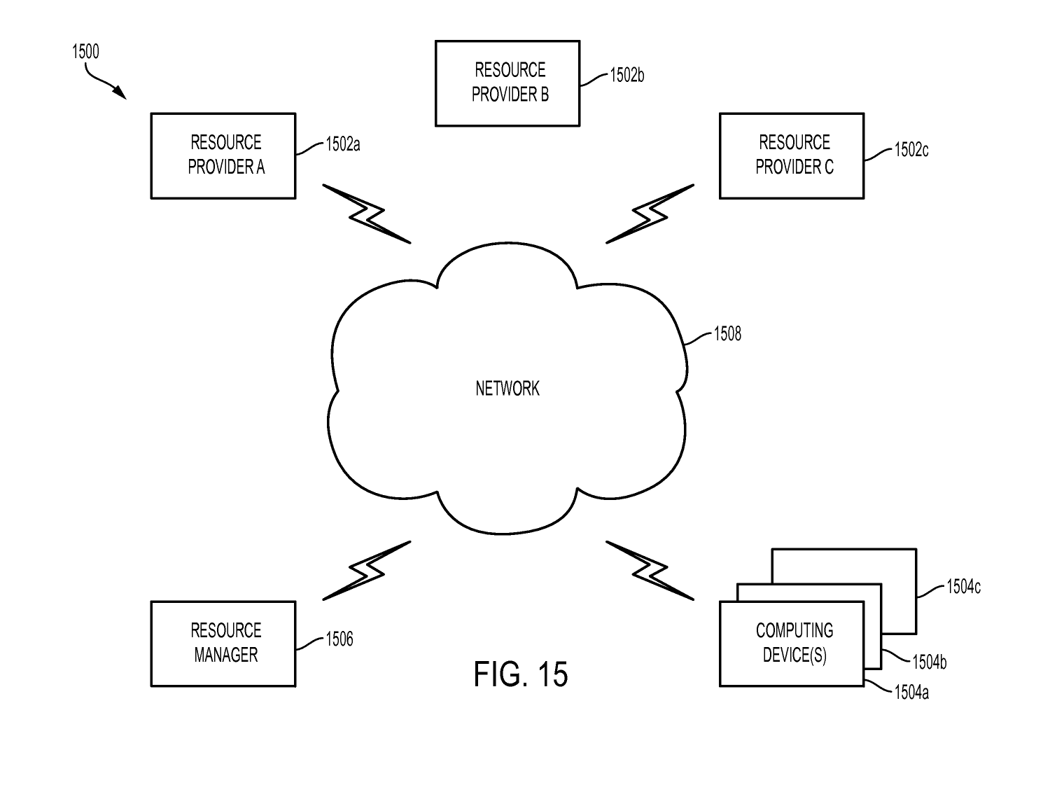

FIG. 15 shows an illustrative network environment 1500 for use in the methods and systems for analysis of spectrometry data corresponding to particles of a sample, as described herein. In brief overview, referring now to FIG. 15, a block diagram of an exemplary cloud computing environment 1500 is shown and described. The cloud computing environment 1500 may include one or more resource providers 1502a, 1502b, 1502c (collectively, 1502). Each resource provider 1502 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1502 may be connected to any other resource provider 1502 in the cloud computing environment 1500. In some implementations, the resource providers 1502 may be connected over a computer network 1508. Each resource provider 1502 may be connected to one or more computing device 1504a, 1504b, 1504c (collectively, 1504), over the computer network 1508.

The cloud computing environment 1500 may include a resource manager 1506. The resource manager 1506 may be connected to the resource providers 1502 and the computing devices 1504 over the computer network 1508. In some implementations, the resource manager 1506 may facilitate the provision of computing resources by one or more resource providers 1502 to one or more computing devices 1504. The resource manager 1506 may receive a request for a computing resource from a particular computing device 1504. The resource manager 1506 may identify one or more resource providers 1502 capable of providing the computing resource requested by the computing device 1504. The resource manager 1506 may select a resource provider 1502 to provide the computing resource. The resource manager 1506 may facilitate a connection between the resource provider 1502 and a particular computing device 1504. In some implementations, the resource manager 1506 may establish a connection between a particular resource provider 1502 and a particular computing device 1504. In some implementations, the resource manager 1506 may redirect a particular computing device 1504 to a particular resource provider 1502 with the requested computing resource.

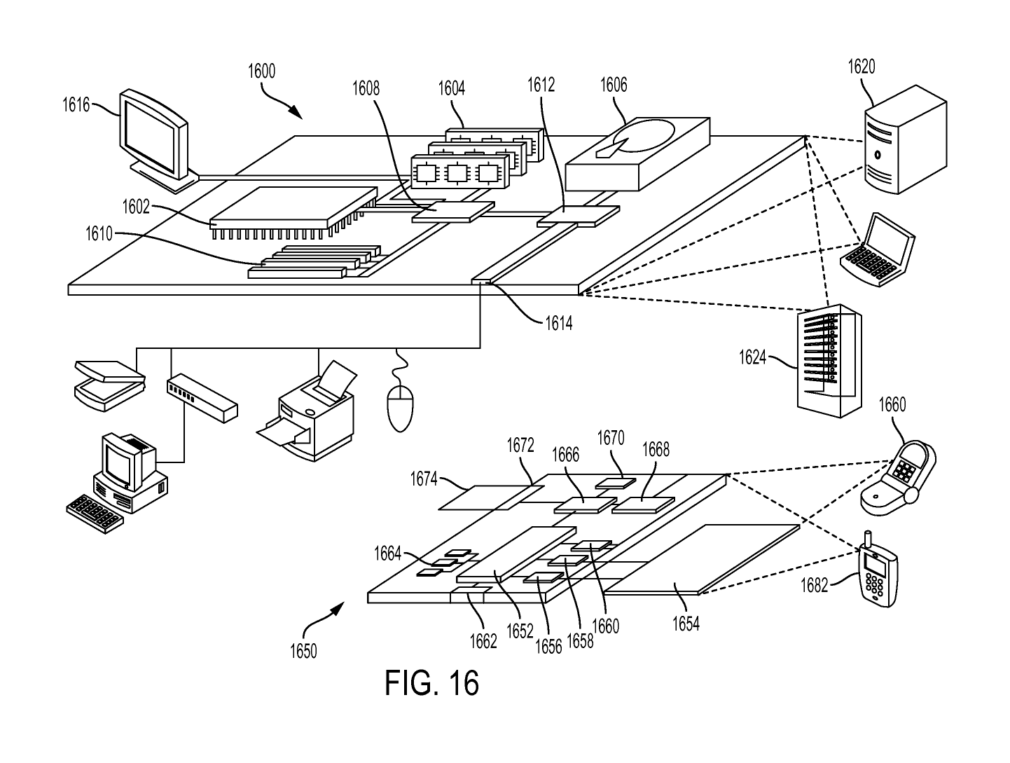

FIG. 16 shows an example of a computing device 1600 and a mobile computing device 1650 that can be used in the methods and systems described in this disclosure. The computing device 1600 is intended to represent various forms of digital computers, such as laptops, desktops, workstations, personal digital assistants, servers, blade servers, mainframes, and other appropriate computers. The mobile computing device 1650 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1600 includes a processor 1602, a memory 1604, a storage device 1606, a high-speed interface 1608 connecting to the memory 1604 and multiple high-speed expansion ports 1610, and a low-speed interface 1612 connecting to a low-speed expansion port 1614 and the storage device 1606. Each of the processor 1602, the memory 1604, the storage device 1606, the high-speed interface 1608, the high-speed expansion ports 1610, and the low-speed interface 1612, are interconnected using various busses, and may be mounted on a common motherboard or in other manners as appropriate. The processor 1602 can process instructions for execution within the computing device 1600, including instructions stored in the memory 1604 or on the storage device 1606 to display graphical information for a GUI on an external input/output device, such as a display 1616 coupled to the high-speed interface 1608. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 1604 stores information within the computing device 1600. In some implementations, the memory 1604 is a volatile memory unit or units. In some implementations, the memory 1604 is a non-volatile memory unit or units. The memory 1604 may also be another form of computer-readable medium, such as a magnetic or optical disk.

The storage device 1606 is capable of providing mass storage for the computing device 1600. In some implementations, the storage device 1606 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1602), perform one or more methods, such as those described above. The instructions can also be stored by one or more storage devices such as computer- or machine-readable mediums (for example, the memory 1604, the storage device 1606, or memory on the processor 1602).

The high-speed interface 1608 manages bandwidth-intensive operations for the computing device 1600, while the low-speed interface 1612 manages lower bandwidth-intensive operations. Such allocation of functions is an example only. In some implementations, the high-speed interface 1608 is coupled to the memory 1604, the display 1616 (e.g., through a graphics processor or accelerator), and to the high-speed expansion ports 1610, which may accept various expansion cards (not shown). In the implementation, the low-speed interface 1612 is coupled to the storage device 1606 and the low-speed expansion port 1614. The low-speed expansion port 1614, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1600 may be implemented in a number of different forms, as shown in the figure. For example, it may be implemented as a standard server 1620, or multiple times in a group of such servers. In addition, it may be implemented in a personal computer such as a laptop computer 1622. It may also be implemented as part of a rack server system 1624. Alternatively, components from the computing device 1600 may be combined with other components in a mobile device (not shown), such as a mobile computing device 1650. Each of such devices may contain one or more of the computing device 1600 and the mobile computing device 1650, and an entire system may be made up of multiple computing devices communicating with each other.

The mobile computing device 1650 includes a processor 1652, a memory 1664, an input/output device such as a display 1654, a communication interface 1666, and a transceiver 1668, among other components. The mobile computing device 1650 may also be provided with a storage device, such as a micro-drive or other device, to provide additional storage. Each of the processor 1652, the memory 1664, the display 1654, the communication interface 1666, and the transceiver 1668, are interconnected using various buses, and several of the components may be mounted on a common motherboard or in other manners as appropriate.

The processor 1652 can execute instructions within the mobile computing device 1650, including instructions stored in the memory 1664. The processor 1652 may be implemented as a chipset of chips that include separate and multiple analog and digital processors. The processor 1652 may provide, for example, for coordination of the other components of the mobile computing device 1650, such as control of user interfaces, applications run by the mobile computing device 1650, and wireless communication by the mobile computing device 1650.

The processor 1652 may communicate with a user through a control interface 1658 and a display interface 1656 coupled to the display 1654. The display 1654 may be, for example, a TFT (Thin-Film-Transistor Liquid Crystal Display) display or an OLED (Organic Light Emitting Diode) display, or other appropriate display technology. The display interface 1656 may comprise appropriate circuitry for driving the display 1654 to present graphical and other information to a user. The control interface 1658 may receive commands from a user and convert them for submission to the processor 1652. In addition, an external interface 1662 may provide communication with the processor 1652, so as to enable near area communication of the mobile computing device 1650 with other devices. The external interface 1662 may provide, for example, for wired communication in some implementations, or for wireless communication in other implementations, and multiple interfaces may also be used.

The memory 1664 stores information within the mobile computing device 1650. The memory 1664 can be implemented as one or more of a computer-readable medium or media, a volatile memory unit or units, or a non-volatile memory unit or units. An expansion memory 1674 may also be provided and connected to the mobile computing device 1650 through an expansion interface 1672, which may include, for example, a SIMM (Single In Line Memory Module) card interface. The expansion memory 1674 may provide extra storage space for the mobile computing device 1650, or may also store applications or other information for the mobile computing device 1650. Specifically, the expansion memory 1674 may include instructions to carry out or supplement the processes described above, and may include secure information also. Thus, for example, the expansion memory 1674 may be provided as a security module for the mobile computing device 1650, and may be programmed with instructions that permit secure use of the mobile computing device 1650. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 1652), perform one or more methods, such as those described above. The instructions can also be stored by one or more storage devices, such as one or more computer- or machine-readable mediums (for example, the memory 1664, the expansion memory 1674, or memory on the processor 1652). In some implementations, the instructions can be received in a propagated signal, for example, over the transceiver 1668 or the external interface 1662.

The mobile computing device 1650 may communicate wirelessly through the communication interface 1666, which may include digital signal processing circuitry where necessary. The communication interface 1666 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1668 using a radio-frequency. In addition, short-range communication may occur, such as using a Bluetooth®, Wi-Fi™, or other such transceiver (not shown). In addition, a GPS (Global Positioning System) receiver module 1670 may provide additional navigation- and location-related wireless data to the mobile computing device 1650, which may be used as appropriate by applications running on the mobile computing device 1650.

The mobile computing device 1650 may also communicate audibly using an audio codec 1660, which may receive spoken information from a user and convert it to usable digital information. The audio codec 1660 may likewise generate audible sound for a user, such as through a speaker, e.g., in a handset of the mobile computing device 1650. Such sound may include sound from voice telephone calls, may include recorded sound (e.g., voice messages, music files, etc.) and may also include sound generated by applications operating on the mobile computing device 1650.

The mobile computing device 1650 may be implemented in a number of different forms, as shown in the figure. For example, it may be implemented as a cellular telephone 1680. It may also be implemented as part of a smart-phone 1682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for identifying a combination of two or more drugs the method comprising the steps of:
(a) performing perturbation experiments whereby cells of a particular type are exposed to combinations of targeted compounds and high-throughput measurements of response profiles are performed to produce phosphoproteomic and/or phenotypic profiles from said perturbation experiments;
(b) automatically extracting, by a processor of a computing device, prior signaling information from one or more databases and generating a qualitative prior model, wherein the qualitative prior model comprises a network of known interactions between proteins of interest;
(c) constructing, by the processor, a network model of signaling using the phosphoproteomic and/or phenotypic profiles from step (a) and the qualitative prior model from step (b);
(d) performing, by the processor, in silico perturbations using the network model of signaling from step (c) to predict responses to perturbation conditions not yet experimentally tested; and
(e) identifying, by the processor, using the predicted responses of step (d), a candidate combination of two or more drugs.

2. The method of claim 1, wherein the combination of two or more drugs is for treatment of cancer.

3. The method of claim 1, wherein the particular type of cells are cancer cells.

4. The method of claim 1, wherein (b) further comprises using a prior extraction and reduction algorithm.

5. The method of claim 1, wherein the proteins of interest are phosphoproteins profiled in step (a).

6. The method of claim 1, wherein the network model of signaling is an ODE-based signaling pathway model.

7. The method of claim 1, wherein the candidate combinations of two or more drugs is for treatment of cancer of the particular type used in the perturbation experiments of step (a).

8. The method of claim 1, further comprising the steps of:
(e) performing additional experimental tests based on the predicted responses of step (d); and
(f) identifying candidate drug combinations based on results of the additional experimental tests in step (e).

9. A method for predicting responses to perturbation conditions to identify candidate drug combinations, the method comprising:
(a) constructing, by a processor of a computing device, a network model of signaling using (i) a phosphoproteomic and/or phenotypic profiles and (ii) a qualitative prior model,
wherein the phosphoproteomic and/or phenotypic profiles having been produced from perturbation experiments in which cells of a particular type are exposed to combinations of targeted compounds and high-throughput measurements of response profiles are performed to produce said phosphoproteomic and/or phenotypic profiles,
and wherein the qualitative prior model comprising a network of known interactions between proteins of interest generated from one or more databases;
(b) performing, by the processor, in silico perturbations using the network model of signaling to predict responses to perturbation conditions not yet experimentally tested; and
(c) identifying, by the processor, a candidate combination of two or more drugs using the predicted responses of step (b).

10. The method of claim 9, wherein the network model of signaling is an ODE-based signaling pathway model.

11. The method of claim 9, wherein the particular type of cells are cancer cells.

12. The method of claim 9, wherein the candidate combination of two or more drugs is for treatment of cancer of the type used in the perturbation experiments.

13. A system for predicting responses to perturbation conditions to identify candidate drug combinations, the system comprising:
a processor; and
a memory having instructions stored thereon, wherein the instructions, when executed by the processor, cause the processor to:
construct a network model of signaling using (i) a phosphoproteomic and/or phenotypic profiles and (ii) a qualitative prior model,
wherein the phosphoproteomic and/or phenotypic profiles having been produced from perturbation experiments in which cells of a particular type are exposed to combinations of targeted compounds and high-throughput measurements of response profiles are performed to produce said phosphoproteomic and/or phenotypic profiles,
and wherein the qualitative prior model comprising a network of known interactions between proteins of interest generated from one or more databases;
perform in silico perturbations using the network model of signaling to predict responses to perturbation conditions not yet experimentally tested; and
identify a candidate combination of two or more drugs using the predicted responses.

14. The system of claim 13, wherein the network model of signaling is an ODE-based signaling pathway model.

15. The system of claim 13, wherein the particular type of cells are cancer cells.

16. The system of claim 13, wherein the candidate combination of two of more drugs is for treatment of cancer of the type used in the perturbation experiments.
